(19) United States
(12) Patent Application Publication
(10) Pub. No.: US 2004/0208843 A1
(43) Pub. Date: Oct. 21, 2004

(54) Cosmetic composition comprising an exogenous ligand-receptor system adsorbed or covalently attached to keratin materials, and hair treatment using this composition or constituent elements thereof

(76) Inventors: Gabin Vic, Venette (FR); Aude Livoreil, Paris (FR); Bruno Bernard, Neuilly Sur Seine (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/667,435

(22) Filed: Sep. 23, 2003

(30) Foreign Application Priority Data
Sep. 24, 2002 (FR) 02 11782

(51) Int. Cl. A61K 7/075; A61K 7/08

(52) U.S. Cl. 424/70.23; 424/70.24; 424/70.27

(57) ABSTRACT

Cosmetic compositions comprising at least one compound (a) and/or at least one complexing agent (b) for complexing the at least one compound (a), and, optionally covalently attached to the at least one compound (a) and/or the at least one complexing agent (b), and optionally at least one cosmetic active agent; processes for treating a keratin material that are based on these compositions, aimed at creating on the keratin material a deposit that gives novel cosmetic properties.

This application claims benefit under 35 U.S.C. §119 of French Application No. FR 0211782, filed in France on Sep. 24, 2002, and which is hereby incorporated by reference.

Disclosed herein are cosmetic compositions comprising at least one compound (a) combined with at least one complexing agent (b), wherein the at least one compound (a) and/or the at least one complexing agent (b) optionally bears at least one cosmetically active group, and/or an anchoring group allowing covalent attachment to a keratin material.

Further disclosed herein are several hair treatment processes based on these compounds, aimed at creating on the fiber a deposit of material that gives novel cosmetic properties.

It can be advantageous to be able to attach onto a keratin material, such as hair, active agents that can provide long-lasting benefits. However, attaching shampoo-resistant cosmetic active agents to the surface of the hair can be difficult to achieve. Among the techniques used at the present time, the simple adsorption of active agents onto the fiber can give effects that may be eliminated at the first shampoo wash, and chemical grafting can pose problems of harmfulness or can cause degradation of the fiber. There is thus still a need to create a deposit of material on the hair that can be shampoo-fast and that does not degrade the fiber.

The present inventors have discovered that by using the forces of association between two compounds exogenous to the hair, cosmetic active agents, bound beforehand to at least one of the compounds, may be deposited onto the surface of the hair.

Disclosed herein are thus cosmetic compositions comprising at least one compound (a) as defined herein and at least one complexing agent (b) capable of forming a complex with the at least one compound (a) having a dissociation constant of less than or equal to 10⁻¹ at a temperature of 25° C.

The at least one compound (a) is optionally linked to an anchoring group a and/or the at least one complexing agent is optionally linked to an anchoring group α′, the groups α and α′, which may be identical or different, allowing covalent attachment to a keratin material and the at least one compound (a) or the at least one complexing agent (b) and optionally bearing at least one cosmetically active group.

Further disclosed herein is the set of cosmetic processes for treating a keratin material, as described herein.

Other embodiments disclosed herein will emerge on reading the description and the examples that follow.

As defined herein, an anchoring group α or α′ is a chemical group that allows the covalent attachment of the compound bearing the anchoring group to the keratin material under the action of a stimulus other than electromagnetic waves with wavelengths ranging from 200 to 800 nm. The stimuli that may be used according to the embodiments disclosed herein may, for example, be chosen from molecular agitation, heat, pH, organic and mineral catalysts, oxygen and ultrasound.

In one embodiment, the at least one compound (a) is chosen from the compounds of formula (I):

L—(A)ₙ—(Y)ₘ—(α)ₚ (I)

wherein:

m is a number ranging from 0 to 100;

n is 0 or 1;

p is a number ranging from 0 to 100;

α is chosen from anchoring groups allowing covalent attachment to a keratin material;

L is chosen from residues, other than biotin, having complexing activity relative to the at least one complexing agent (b); L may for example, be chosen from, antigens, carbohydrates, nucleic acid monostrands, and enzyme substrates among other things;

A is chosen from linear and branched, saturated and unsaturated divalent radicals comprising from 1 to 100 carbon atoms, optionally interrupted with at least one hetero atom and optionally substituted with at least one group chosen from hydroxyl, amino, halogen, aryl, phosphate, phosphonate, sulphate, sulphonate, carboxyl, alkoxycarbonyl and alkoxy groups;

Y is chosen from groups Z, wherein Z is chosen from carboxyl, mercaptan, alkoxycarbonyl, amino, ammonio, phosphate, phosphite, phosphonate, sulphite, sulphate and sulphonate groups and from radicals BW, wherein W is chosen from residues with cosmetic activity and B is chosen from a species for fixing the residue W to L or to A,

B is chosen, for example, from amine, imine, amide, ester, disulphide, thioester, urethane, urea, ether, thioether, azo, methine and epoxy groups, aromatic and heterocyclic residues and dialkylpolysiloxane residues; and

W is derived, for example, from at least one of: UV-screening molecules, hydrating and emollient molecules, conditioners, antistatic agents, antiperspirants, fragrancing materials, reducing molecules, oxidizing molecules, coloring molecules, antimicrobial agents, antidandruff agents, mineral and organic particulates onto which are optionally adsorbed at least one polymer, and anionic, nonionic, amphoteric and cationic film-forming agents onto which are optionally adsorbed organic and/or mineral particles.

As used herein, the term “particles” covers both mineral particles and organic particles. The mineral particles may, for example, be chosen from oxides, oxide dihydrates, hydroxides, carbonates, sulphides, silicates and phosphates of silicon, of calcium, of magnesium, of zinc, of aluminium, of titanium, of zirconium and of cerium, nacres, micas, particles comprising native metals such as alkali metals, alkaline-earth metals, transition metals and rare-earth metals, which may be optionally coated, and alloys thereof. These metals may, for example, be chosen from aluminium, copper, silver, gold, indium, iron, platinum, nickel, molybdenum, silicon, titanium, tungsten, antimony, palladium, cadmium, zinc, tin and selenium, and alloys thereof. In one embodiment, the metals may, for example, be chosen from gold, silver, palladium and platinum and alloys thereof. Further, the metals may, for example, be chosen from silver and gold and alloys thereof.

The organic particles may, for example, be chosen from latices, polystyrene, polystyrene derivatives, silicones, fluoro polymers, polypropylene, polyethylene, poly(meth)acrylic acid, polymethacrylate, polyurethane, polyamide, polycarbonate, polyvinyl chloride, polyvinyl acetate, fluoropolymers, polyethylenes, polypropylene, polyisobutylene, poly(1-butylene), copolymers and blends of the polymers mentioned, alkyl cellulose, hydroxyalkyl cellulose, cellulose ethers, cellulose esters, hydroxypropylcellulose, hydroxypropyldextran, hydroxypropylmethyl cellulose, cellulose acetate, carboxyethylcellulose, cellulose sulphate, dextran sulphate, polyvinyl alcohol, polyethylene oxide, polyvinyl chloride and polyvinylpyrrolidone.

The polystyrene particles are commercially available from Polyscience Inc. (Warrington, Pa.) or Duke Scientific Corporation (Palo Alto, Ca.).

These organic particles may be crosslinked with at least one crosslinking agent. For example, the at least one crosslinking agent may be chosen from divinylbenzene, glutaraldehyde, 1,4-bis(acryloyl)piperazine, carbodiimides, N-hydroxysuccinimide and derivatives thereof, divinyl sulphone, dithiobis(succinimidyl)propioniate and N-succinimidyl-3-(2-pyridyldithio)propionate.

As defined herein, the term “particles” means particles ranging, for example, from 1 nm to 100 μm in size, further, for example, from 1 nm to 1000 nm in size and even further, for example, from 1 nm to 50 nm in size. The term “particle size” means the maximum possible size to measure between two diametrically opposite points of the particle.

The particles disclosed herein may, for example, be chosen from spherical, lamellar, fibrillar, and totally random shaped particles.

The group W may, for example, be derived from at least one active agent chosen from:

modified or unmodified, optionally hydrolysed saccharides, oligosaccharides and polysaccharides;

amino acids, modified or unmodified, optionally hydrolysed oligopeptides, peptides, and proteins, polyamino acids and enzymes;

branched or unbranched fatty alcohols and fatty acids;

animal, plant and mineral waxes;

ceramides and pseudoceramides;

hydroxylated organic acids;

UV screening agents;

antioxidants and free-radical scavengers;

chelating agents;

antidandruff agents;

seborrhoea regulators;

calmants;

cationic surfactants;

cationic polymers;

amphoteric polymers;

optionally organomodified silicones;

mineral, plant and animal oils;

polyisobutenes and poly(α-olefins);

esters;

soluble or dispersed anionic polymers;

soluble or dispersed nonionic polymers

reducing agents; and

dyes.

For example, the modified or unmodified, optionally hydrolysed saccharides, oligosaccharides and polysaccharides which may be used in the compositions disclosed herein, may be chosen from those described, for example, in “Encyclopedia of Chemical Technology”, Kirk-Othmer, Third Edition, 1982, volume 3, pp. 896-900 and volume 15, pp. 439-458, in “Polymers in Nature” by E. A. MacGregor and C. T. Greenwood, published by John Wiley & Sons, Chapter 6, pp. 240-328, 1980, and in “Industrial Gums—Polysaccharides and their Derivatives”, edited by Roy L. Whistler, Second Edition, published by Academic Press Inc.

The modified or unmodified, optionally hydrolysed saccharides, oligosaccharides and polysaccharides that may be used in the compositions disclosed herein may, for example, be chosen from glucans; modified or unmodified starches (such as those derived, for example, from cereals, such as wheat, corn and rice, from vegetables, such as haricot beans, and from tubers, such as potato and cassava), which are different from the starch betain as described above, amylose, amylopectin, glycogen, dextrans, β-glucans, celluloses and derivatives thereof (such as methylcelluloses, hydroxyalkylcelluloses, ethylhydroxyethylcelluloses and carboxymethylcelluloses), fructosans, inulin, levane, mannans, xylans, lignins, arabans, galactans, galacturonans, chitin, glucoronoxylans, arabinoxylans, xyloglucans, galactomannans, glucomannans, pectic acids and pectins, alginic acid and alginates, arabinogalactans, carrageenans, agars, glycosaminoglucans, gum arabics, gum tragacanths, ghatti gums, karaya gums, carob gums, guar gums and xanthan gums.

The amino acids may, for example, be chosen from at least one of cysteine, lysine, alanine, N-phenylalanine, arginine, glycine, and leucine.

The modified or unmodified, optionally hydrolysed oligopeptides, peptides and proteins that may be used in the compositions disclosed herein may, for example, be chosen from modified and unmodified wools, silk protein hydrolysates, and plant proteins such as wheat proteins.

The polyamino acids that may be used in the compositions disclosed herein may, for example, be polylysine.

The enzymes that may be used herein may, for example, be chosen from laccases, peroxidases, lipases, proteases, glycosidases, dextranases, uricases and alkaline phosphatase.

The branched or unbranched fatty acids that are suitable for use in the compositions disclosed herein may, for example, be chosen from at least one of C₈-C₃₀ carboxylic acids such as palmitic acid, oleic acid, linoleic acid, myristic acid, stearic acid and lauric acid. The fatty alcohols that may be used in the compositions disclosed herein may, for example, be chosen from at least one of C₈-C₃₀ alcohols such as, palmityl alcohol, oleyl alcohol, linoleyl alcohol, myristyl alcohol, stearyl alcohol and lauryl alcohol.

As used herein, a wax is a lipophilic compound that is solid at room temperature (about 25° C.), with a reversible solid/liquid change of state, having a melting point of greater than 40° C. and which may be up to 200° C., and having an anisotropic crystal organization in the solid state. In general, the size of the wax crystals is such that crystals scatter and/or diffuse light, giving the compositions comprising them a more or less opaque, cloudy appearance. By raising the wax to its melting point, it is possible to make it miscible with oils and to form a microscopically homogeneous mixture, but upon returning the temperature of the mixture to room temperature, a microscopically and macroscopically detectable recrystallization of the wax in the oils of the mixture is obtained (opalescence).

The waxes used in the compositions disclosed herein may, for example, be chosen from waxes of animal origin such as beeswax, spermaceti, lanolin wax and lanolin derivatives; plant waxes such as carnauba wax, candelilla wax, ouricury wax, Japan wax, cocoa butter, cork fiber wax and sugar cane wax; and mineral waxes, such as paraffin wax, petroleum jelly wax, lignite wax, microcrystalline waxes and ozokerites.

The ceramides useful herein may, for example, be chosen from ceramides of categories I, II, III and V according to the Dawning classification, for example, N-oleyldehydrosphingosine.

The hydroxylated organic acids may, for example, be chosen from those that are well known and used in the art. For example, the hydroxylated organic acids may be chosen from citric acid, lactic acid, tartaric acid and malic acid.

The UV-A-active and UV-B-active sunscreens that may be used in the compositions disclosed herein may be chosen from those that are well known to those skilled in the art. For example, the UV-A-active and UV-B-active sunscreens may be chosen from dibenzoylmethane derivatives such as 4-methyldibenzoylmethane, 4-isopropyldibenzoylmethane, 4-tert-butyldibenzolymethane, 2,4-dimethyldibenzoylmethane, and 4-tert-butyl-4′-diisopropyldibenzoylmethane; p-aminobenzoic acid and esters thereof, such as 2-ethylhexyl p-dimethylaminobenzoate and N-propoxylated ethyl p-aminobenzoate; salicylates, such as triethanolamine salicylate; cinnamic acid esters, such as 2-ethylhexyl 4-methoxycinnamate and methyl diisopropylcinnamate; methyl anthranilate; benzotriazole derivatives; triazine derivatives; β,β′-diphenylacrylate derivatives, such as 2-ethylhexyl 2-cyano-3,3-diphenylacrylate and ethyl 2-cyano-3,3-diphenylacrylate; 2-phenylbenzimidazole-5-sulphonic acid and salts thereof; benzophenone derivatives; benzylidenecamphor derivatives; and silicone screening agents, etc.

The antioxidants and free-radical scavengers that may be used in the compositions disclosed herein may, for example, be chosen from ascorbic acid; ascorbyl compounds such as ascorbyl dipalmitate; t-butylhydroquinone; polyphenols, such as phloroglucinol; sodium sulphite; erythorbic acid; and flavonoids.

Useful chelating agents may be chosen, for example, from EDTA (ethylenediaminetetraacetic acid) and salts thereof, such as disodium EDTA and dipotassium EDTA; phosphate compounds, such as sodium metaphosphate, sodium hexametaphosphate and tetrapotassium pyrophosphate; and phosphonic acids and salts thereof, such as ethylenediaminetetramethylenephosphonic acid salts.

Useful antidandruff agents may be chosen, for example, from:

benzethonium chloride, benzalkonium chloride, chlorhexidine, chloramine T, chloramine B, 1,3-dibromo-5,5-dimethylhydantoin, 1,3-dichloro-5,5-dimethylhydantoin, 3-bromo-1-chloro-5,5-dimethylhydantoin and N-chlorosuccinimide;

1-hydroxy-2-pyridone derivatives, such as 1-hydroxy-4-methyl-2-pyridone, 1-hydroxy-6-methyl-2-pyridone and 1-hydroxy-4,6-dimethyl-2-pyridone;

trihalocarbamides;

triclosan;

azole compounds, such as climbazole, ketoconazole, clotrinazole, econazole, isoconazole and miconazole b;

antifungal polymers, such as amphotericin B and nystatin;

selenium sulphides; and

sulphur in its various forms, cadmium sulphide, allantoin, coal tar and wood tar and, for example, derivatives thereof, cade oil, undecylenic acid, fumaric acid and allylamines, such as terbinafine.

The antidandruff agents may also, for example, be chosen from the addition salts thereof with physiologically acceptable acids, for example, the addition salts may, for example, be chosen from sulphuric acid, nitric acid, thiocyanic acid, hydrochloric acid, hydrobromic acid, hydriodic acid, phosphoric acid, acetic acid, benzoic acid, glycolic acid, aceturic acid, succinic acid, nicotinic acid, tartaric acid, maleic acid, palmitic acid, methanesulphonic acid, propanoic acid, 2-oxopropanoic acid, propanedioic acid, 2-hydroxy-1,4-butanedioic acid, 3-phenyl-2-propenoic acid, α-hydroxybenzeneacetic acid, ethanesulphonic acid, 2-hydroxyethanesulphonic acid, 4-methylbenzenesulphonic acid, 4-amino-2-hydroxybenzoic acid, 2-phenoxybenzoic acid, 2-acetyloxybenzoic acid, picric acid, lactic acid, citric acid, malic acid, oxalic acid and amino acid salts.

The antidandruff agents mentioned above may also, where appropriate, be chosen from the addition salts thereof with physiologically acceptable organic and mineral bases. The organic bases may, for example, be chosen from low molecular weight alkanolamines, such as ethanolamine, diethanolamine, N-ethylethanolamine, triethanolamine, diethylaminoethanol and 2-amino-2-methylpropanedione; non-volatile bases, such as ethylenediamine, hexamethylenediamine, cyclohexylamine, benzylamine and N-methylpiperazine; quaternary ammonium hydroxides, for example, trimethylbenzyl hydroxide; guanidine and derivatives thereof, for example, alkyl derivatives thereof. The mineral bases may, for example, be chosen from alkali metal salts, such as the sodium and potassium salts; the ammonium salts, the alkaline-earth metal salts, for example, the magnesium and calcium salts; the salts of cationic di-, tri- and tetravalent metals, for example, the zinc, aluminium and zirconium salts. Alkanolamines, ethylenediamine and mineral bases such as the alkali metal salts may, for example, be used in the compositions disclosed herein.

The seborrhoea regulators may, for example, be chosen from succinyl-chitosan and poly-β-alanine.

The calmants may, for example, be chosen from azulene and glycyrrhetinic acid.

The cationic surfactants may, for example, be chosen from those that are well known per se, such as optionally polyoxyalkylenated primary, secondary and tertiary fatty amine salts; quaternary ammonium salts such as tetraalkylammonium, alkylamido-alkyltrialkylammonium, trialkylbenzylammonium, trialkylhydroxyalkylammonium and alkylpyridinium chlorides and bromides; and imidazoline derivatives.

As defined herein, the term “cationic polymer” means any polymer comprising at least one group chosen from cationic groups and groups that may be ionized into cationic groups.

The cationic polymers that may be used in the compositions disclosed herein may, for example, be chosen from any of those already known per se for improving the cosmetic properties of hair treated with detergent compositions, such as those described in Patent Application No. EP-A-0 337 354 and in French Patent Application Nos. FR-A-2 270 846, 2 383 660, 2 598 611, 2 470 596 and 2 519 863.

The cationic polymers may, for example, be chosen from polymers comprising units comprising at least one group chosen from primary, secondary, tertiary and quaternary amine groups and the units may either form part of the main polymer chain and/or may be borne by a side substituent that is directly attached thereto.

The cationic polymers used may, for example, have a number-average molecular mass ranging from 500 to 5×10⁶ and further, for example, from 10³ to 3×10⁶.

The cationic polymers may, for example, be chosen from polyamine, polyamino amide and polyquaternary ammonium type polymers. These polymers are known products.

The polyamine, polyamino amide and polyquaternary ammonium type polymers that may be used in the compositions disclosed herein may, for example, be chosen from those described in French Patent Nos. 2 505 348 and 2 542 997. For example, these polymers may be chosen from at least one of:

(1) homopolymers and copolymers derived from acrylic acid, methacrylic acid esters and amides;

(2) cellulose ether derivatives comprising at least one quaternary ammonium group, described in French Patent No.1 492 597;

(3) cationic cellulose derivatives, such as cellulose copolymers and cellulose derivatives grafted with a water-soluble quaternary ammonium monomer, and described, for example, in U.S. Pat. No. 4,131,576, such as hydroxyalkylcelluloses, for example, hydroxymethyl-, hydroxyethyl- and hydroxypropylcelluloses grafted, for example, with a methacryloylethyltrimethylammonium, methacrylamidopropyltrimethylammonium and dimethyldiallylammonium salt;

(4) cationic polysaccharides described, for example, in U.S. Pat. Nos. 3,589,578 and 4,031,307, such as guar gums comprising at least one cationic trialkylammonium group;

(5) polymers comprising at least one piperazinyl unit and at least one group chosen from divalent alkylene and hydroxyalkylene groups, wherein the divalent alkylene and hydroxyalkylene groups comprise at least one chain chosen from straight and branched chains, optionally interrupted by at least one oxygen, sulphur or nitrogen atom; aromatic and heterocyclic rings; and the oxidation and quaternization products of these polymers. Such polymers are described, for example, in French Patent Nos. 2 162 025 and 2 280 361;

(6) water-soluble polyamino amides such as those described, for example, in French Patent Nos. 2 252 840 and 2 368 508;

(7) polyamino amide derivatives, for example, chosen from adipic acid/dialkylaminohydroxyalkyldialkylenetriamine polymers wherein the alkyl group comprises from 1 to 4 carbon atoms and, for example, is chosen from methyl, ethyl and propyl groups, and the alkylene group comprises from 1 to 4 carbon atoms and, for example, may be an ethylene group. Such polymers are described, for example, in French Patent No.1 583 363.

(8) polymers obtained by reaction of a polyalkylene polyamine comprising two primary amine groups and at least one secondary amine group with a dicarboxylic acid chosen from diglycolic acid and saturated aliphatic dicarboxylic acids comprising from 3 to 8 carbon atoms, wherein the molar ratio of the polyalkylene polyamine to the dicarboxylic acid ranges from 0.8:1 to 1.4:1. The polyamino amide resulting therefrom is reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2,961,347;

(9) cyclopolymers of alkyldiallylamine and of dialkyldiallylammonium such as the dimethyldiallylammonium chloride homopolymer and copolymers of diallyldimethylammonium chloride and of acrylamide;

(10) quaternary diammonium polymers with a number-average molecular mass ranging from 1000 to 100 000 such as those described, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020;

(11) polyquaternary ammonium polymers such as those described in Patent Application No. EP-A-122 324;

(12) quaternary polymers of vinylpyrrolidone and of vinylimidazole, such as, the products sold under the names Luviquat® FC 905, FC 550 and FC 370 by the company BASF;

(13) polyamines such as Polyquart® H sold by Henkel under the reference name “Polyethylene glycol (15) tallow polyamine” in the CTFA dictionary; and

(14) crosslinked (meth)acryloyloxy(C₁-C₄)alkyltri(C₁-C₄)alkylammonium salt polymers such as those sold under the name Salcare® SC 92, Salcare® SC 95 and Salcare® SC 96 by the company Allied Colloids.

Other cationic polymers that may be used in the compositions disclosed herein may, for example, be chosen from cationic proteins and cationic protein hydrolysates, polyalkyleneimines, such as polyethyleneimines; polymers comprising at least one unit chosen from vinylpyridine and vinylpyridinium units; condensates of polyamines and of epichlorohydrin; quaternary polyureylenes; and chitin derivatives.

The amphoteric polymers that may be used in the compositions disclosed herein may, for example, be chosen from polymers comprising units B and C randomly distributed in the polymer chain, wherein B is unit derived from a monomer comprising at least one basic nitrogen atom and C is a unit derived from an acidic monomer comprising at least one group chosen from carboxylic and sulphonic groups, or alternatively B and C may be chosen from groups derived from zwitterionic carboxybetaine and sulphobetaine monomers;

B and C may also, for example, be chosen from cationic polymer chains comprising at least one group chosen from primary, secondary, tertiary and quaternary amine groups, wherein at least one of the amine groups bears at least one group chosen from carboxylic and sulphonic groups linked via a hydrocarbon-based radical, or alternatively B and C form part of a chain of a polymer comprising at least one ethylene-dicarboxylic unit wherein at least one of the carboxylic groups has been made to react with a polyamine comprising at least one group chosen from primary and secondary amine groups.

The amphoteric polymers corresponding to the above definition may, for example, be chosen from the following polymers:

(1) polymers resulting from the copolymerization of at least one monomer derived from a vinyl compound bearing at least one carboxylic group such as, acrylic acid, methacrylic acid, maleic acid, alpha-chloroacrylic acid, and at least one basic monomer derived from a substituted vinyl compound comprising at least one basic atom, such as, dialkylaminoalkyl methacrylate and acrylate, dialkylaminoalkylmethacrylamide and acrylamide. Such compounds are described in U.S. Pat. No. 3,836,537.

The amphoteric polymers used in the compositions disclosed herein may, for example, be the sodium acrylate/acrylamidopropyl- trimethylammonium chloride copolymer sold under the name Polyquart® KE 3033 by the company Henkel.

The vinyl compound may also be a dialkyldiallylammonium salt such as dimethyldiallylammonium chloride.

The copolymers of acrylic acid and of the latter monomer are sold under the names Merquat® 280, Merquat® 295 and Merquat® Plus 3330 by the company Calgon.

(2) polymers comprising units derived from:

a) at least one monomer chosen from acrylamides and methacrylamides substituted on the nitrogen with an alkyl radical,

b) at least one acidic comonomer comprising at least one reactive carboxylic group, and

c) at least one basic comonomer, such as esters comprising at least one substituent chosen from primary, secondary, tertiary and quaternary amine substituents of acrylic and methacrylic acids and the product of quaternization of dimethylaminoethyl methacrylate with dimethyl and diethyl sulphate.

The N-substituted acrylamides and methacrylamides which may be used in the compositions disclosed herein may, for example, be chosen from groups wherein the alkyl radicals comprise from 2 to 12 carbon atoms, for example, N-ethylacrylamide, N-tert-butylacrylamide, N-tert-octylacrylamide, N-octylacrylamide, N-decylacrylamide, N-dodecylacrylamide and the corresponding methacrylamides.

The at least one acidic comonomer may, for example, be chosen from acrylic acids, methacrylic acids, crotonic acids, itaconic acids, maleic acids, fumaric acids, and alkyl monoesters, comprising from 1 to 4 carbon atoms, of maleic and fumaric acids and anhydrides. The at least one basic comonomer may, for example, be chosen from aminoethyl, butylaminoethyl, N,N′-dimethylaminoethyl and N-tert-butylaminoethyl methacrylates. The copolymers whose CTFA (4th edition, 1991) name is octylacrylamide/acrylates/butylaminoethyl methacrylate copolymer such as the products sold under the name Amphomer® and Lovocryl® 47 by the company National Starch may also, for example, be used in the compositions disclosed herein.

(3) crosslinked and alkylated polyamino amides partially or totally derived from polyamino amides of general formula:

)CO—R₁₀—CO—Z] (II)

wherein:

R₁₀ is a divalent radical derived from a saturated dicarboxylic acid, amono- or dicarboxylic aliphatic acid comprising at least one ethylenic double bond, an ester of a lower alkanol, comprising from 1 to 6 carbon atoms, of these acids, and radicals derived from the addition of any one of the acids to amines chosen from bis(primary) and bis(secondary) amines; and

Z is chosen from bis(primary), mono- and bis(secondary) polyalkylene-polyamine radicals and may, for example, be chosen from radicals of the following formula:

a) in proportions ranging from 60 to 100 mol %, the radical

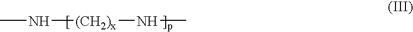

wherein: x=2 and p=2 or 3, or alternatively x=3 and p=2

this radical being derived from a compound chosen from diethylenetriamine, triethylenetetraamine and dipropylenetriamine;

b) in proportions ranging from 0 to 40 mol %, the radical (III) above wherein x=2 and p=1, wherein the radical is derived from a compound chosen from ethylenediamine and piperazine:

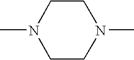

c) in proportions ranging from 0 to 20 mol %, the —NH—(CH₂)₆—NH— radical derived from hexamethylenediamine, these polyamino amines being crosslinked by addition of at least one difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

The saturated carboxylic acids may, for example, be chosen from acids comprising from 6 to 10 carbon atoms, such as adipic acid, 2,2,4-trimethyladipic acid and 2,4,4-trimethyladipic acid, terephthalic acid, and acids comprising at least one ethylenic double bond, such as acrylic acid, methacrylic acid and itaconic acid. The alkane sultones used in the alkylation may, for example, be chosen from propane sultone and butane sultone, and the salts of the alkylating agents may, for example, be chosen from sodium and potassium salts.

(4) polymers comprising at least one zwitterionic unit of formula:

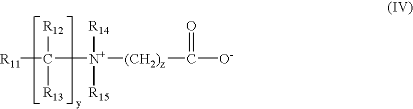

wherein:

R₁₁ is chosen from polymerizable unsaturated groups, such as acrylate, methacrylate, acrylamide and methacrylamide groups;

y and z, which may be identical or different, are integers ranging from 1 to 3;

R₁₂ and R₁₃, which may be identical or different, are each chosen from a hydrogen atom, and methyl, ethyl and propyl groups,

R₁₄ and R₁₅, which may be identical or different, are each chosen from a hydrogen atom and alkyl radicals such that the sum of the carbon atoms in R₁₄ and R₁₅ does not exceed 10.

The polymers comprising at least one zwitterionic unit can also comprise at least one unit derived from non-zwitterionic monomers, such as monomer chosen from dimethyl and diethylaminoethyl acrylates and methacrylates, alkyl acrylates, methacrylates, acrylamides, methacrylamides, and vinyl acetate.

For example, the copolymer of methyl methacrylate/dimethyl carboxymethylammonio ethyl methacrylate such as the product sold under the name Diaformer® Z301 by the company Sandoz can be used in the compositions disclosed herein.

5) polymers derived from chitosan comprising monomer units corresponding to the formulae (V), (VI), and (VII) below:

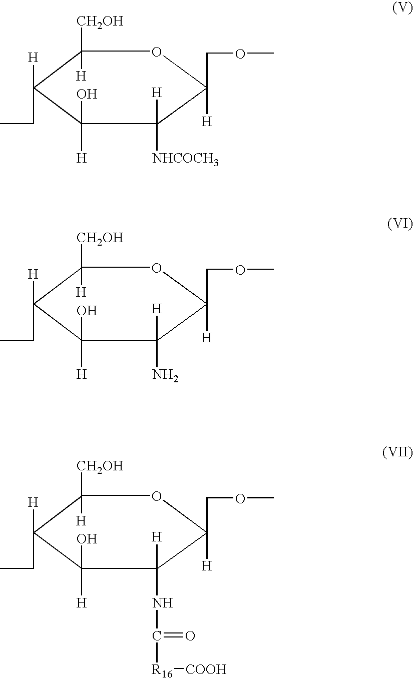

the unit (V) being present in proportions ranging from 0 to 30%, the unit (VI) in proportions ranging from 5 to 50% and the unit (VII) in proportions ranging from 30 to 90%, and wherein in the unit (VII), R₁₆ is a radical of formula:

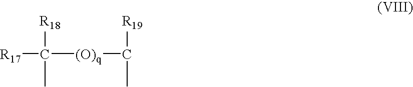

wherein:

if q=0, R₁₇, R₁₈ and R₁₉, which may be identical or different, each are chosen from a hydrogen atom, methyl, hydroxyl, acetoxy and amino residues; monoalkylamine residues and dialkylamine residues which are optionally interrupted by at least one nitrogen atom and/or optionally substituted with at least one group chosen from amine, hydroxyl, carboxyl, alkylthio and sulphonic groups; alkylthio residues wherein the alkyl radical bears at least one amino residue, at least one of the radicals R₁₇, R₁₈ and R₁₉ being, in this case, a hydrogen atom; or

if q=1, R₁₇, R₁₈ and R₁₉, which may be identical or different, are each chosen from a hydrogen atom and the salts formed by these compounds with bases or acids.

(6) polymers derived from the N-carboxyalkylation of chitosan, such as N-carboxymethylchitosan and N-carboxybutylchitosan sold under the name Evalsan® by the company Jan Dekker.

(7) polymers corresponding to the general formula (IX) as described, for example, in French Patent No. 1 400 366:

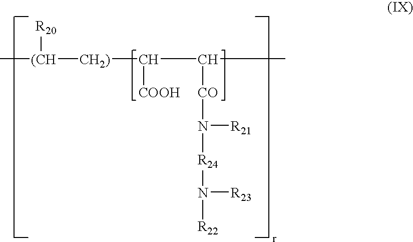

wherein:

R₂₀ is chosen from a hydrogen atom, a CH₃O radical, a CH₃CH₂O radical, and a phenyl radical;

R₂₁ is chosen from a hydrogen atom and lower alkyl radicals, such as methyl and ethyl radicals;

R₂₂ is chosen from a hydrogen atom and lower alkyl radicals, such as methyl and ethyl radicals;

R₂₃ is chosen from lower alkyl radicals, such as methyl and ethyl radicals and radicals corresponding to the formula: —R₂₄—N(R₂₂)₂, wherein R₂₄ is chosen from —CH₂—CH₂—, —CH₂—CH₂—CH₂— and —CH₂—CH(CH₃)— groups, R₂₂ is chosen from hydrogen and lower alkyl radicals such as methyl and ethyl and the higher homologues of these radicals comprising up to 6 carbon atoms; and

r is a number ranging from 1 to 100.

(8) amphoteric polymers of the type -D-X-D-X- chosen from:

a) polymers obtained by the action of chloroacetic acid or sodium chloroacetate on compounds comprising at least one unit of formula:

-D-X-D-X-D- (X)

wherein:

D is a radical

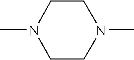

and;

X is chosen from E and E′, wherein E and E′, which may be identical or different, are each chosen from divalent alkylene radicals which comprise at least one straight or branched chain comprising up to 7 carbon atoms in the main chain, which are optionally substituted with at least one hydroxyl group and which can additionally comprise at least one oxygen, nitrogen or sulphur atom and 1 to 3 aromatic and/or heterocyclic rings; wherein the oxygen, nitrogen and sulphur atoms can be present in the form of at least one group chosen from ether, thioether, sulphoxide, sulphone, sulphonium, alkylamine, and alkenylamine groups, hydroxyl, benzylamine, amine oxide, quaternary ammonium, amide, imide, alcohol, ester and urethane groups;

b) polymers of formula:

-D-X-D-X- (XI)

wherein:

D is a radical

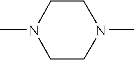

and;

X is chosen from the E and E′ and wherein at least one X is chosen from E′; E having the meaning given above and E′ being chosen from divalent alkylene radicals comprising at least one chain chosen from straight and branched chains comprising up to 7 carbon atoms in the main chain, wherein the divalent alkylene radicals are optionally substituted with at least one hydroxyl radical and comprise at least one nitrogen atom, the at least one nitrogen atom being substituted with an alkyl chain which is optionally interrupted by an oxygen atom, and wherein the alkyl chain comprises at least one functional group chosen from carboxyl functional groups and hydroxyl functional groups, and wherein the alkyl chain is betainized by reaction with a reactant chosen from chloroacetic acid and sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also comprise other vinyl comonomers such as vinylcaprolactam.

The silicones that may be used in the compositions disclosed herein may be soluble or insoluble in water, and they may, for example, be chosen from organopolysiloxanes that are insoluble in water; the silicones may, further, for example, be in the form of oils, waxes, resins, or gums.

The organopolysiloxanes are defined in greater detail in Walter Noll's “Chemistry and Technology of Silicones” (1968) Academic Press. The organopolysiloxanes can be chosen from volatile and non-volatile organopolysiloxanes.

When they are volatile, the organopolysiloxanes may, for example, be chosen from those having a boiling point ranging from 60° C. to 260° C., and further, for example, from:

(i) cyclic silicones comprising, for example, from 3 to 7 silicone atoms, and further, for example, from 4 to 5 silicon atoms. These cyclic silicones may, for example, be chosen from octamethylcyclotetrasiloxane sold, for example, under the name “Volatile Silicone 7207” by Union Carbide and “Silbione 70045 V 2” by Rhodia, and decamethylcyclopentasiloxane sold under the name “Volatile Silicone 7158” by Union Carbide, and “Silbione 70045 V 5” by Rhodia.

The cyclic silicones may also be chosen from dimethylsiloxane/methylalkylsiloxane type cyclocopolymers, such as “Volatile Silicone FZ 3109” sold by the company Union Carbide, having the chemical structure:

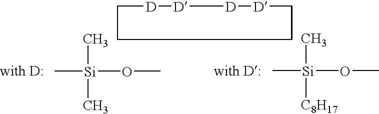

(ii) linear volatile silicones comprising from 2 to 9 silicon atoms and having a viscosity of less than or equal to 5×10⁻⁶ m²/s at 25° C. For example, decamethyltetrasiloxane sold, for example, under the name “SH 200” by the company Toray Silicone can be used. Silicones chosen from this category are also described in the article published in Cosmetics and Toiletries, Vol. 91, January 76, pp. 27-32, Todd & Byers “Volatile Silicone Fluids for Cosmetics”.

The non-volatile silicones may, for example, be chosen from polyalkylsiloxanes, polyarylsiloxanes, polyalkylarylsiloxanes, silicone gums and resins, and polyorganosiloxanes modified with at least one organofunctional group.

The organomodified silicones that may be used in the compositions disclosed herein are silicones as defined above and comprising in their structure at least one organofunctional group attached via a hydrocarbon-based group.

The organomodified silicones may, for example, be chosen from polyorganosiloxanes comprising at least group chosen from:

polyethyleneoxy and polypropyleneoxy groups optionally comprising at least one alkyl group chosen from C₆-C₂₄ alkyl groups, such as the products known as dimethicone copolyol sold by the company Dow Corning under the name DC 1248 and the oils Silwet® L 722, L 7500, L 77 and L 711 from the company Union Carbide and the (C₁₂)alkylmethicone copolyol sold by the company Dow Corning under the name Q2 5200;

substituted and unsubstituted amine groups, such as the products sold under the name GP 4 Silicone Fluid and GP 7100 by the company Genesee, and the products sold under the names Q2 8220 and Dow Corning 929 or 939 by the company Dow Corning. The substituted amine groups may, for example, be chosen from C₁-C₄ aminoalkyl groups;

thiol groups such as the products sold under the names “GP 72 A” and “GP 71” from Genesee;

alkoxylated groups such as the product sold under the name “Silicone Copolymer F-755” by SWS Silicones and Abil Wax® 2428, 2434 and 2440 by the company Goldschmidt;

hydroxylated groups, such as the polyorganosiloxanes comprising at least one hydroxyalkyl functional group, described in French Patent Application No. FR-A-85/16334;

acyloxyalkyl groups, such as the polyorganosiloxanes described in U.S. Pat. No. 4,957,732;

anionic groups of the carboxylic acid type, such as the products described in Patent No. EP 186 507 from the company Chisso Corporation, and anionic groups of alkylcarboxylic type, such as those present in the product X-22-3701E from the company Shin-Etsu; 2-hydroxyalkyl sulphonate; 2-hydroxyalkyl thiosulphate such as the products sold by the company Goldschmidt under the names “Abil® S201” and “Abil® S255;” and

hydroxyacylamino groups, such as the polyorganosiloxanes described in Patent Application No. EP 342 834, for example, the product Q2-8413 from the company Dow Corning.

The compositions disclosed herein may further comprise at least one oil of plant, mineral, or animal origin. The oils of plant origin may be chosen from, for example, sweet almond oil, avocado oil, castor oil, olive oil, jojoba oil, sunflower oil, wheatgerm oil, sesame seed oil, groundnut oil, grapeseed oil, soybean oil, rapeseed oil, safflower oil, coconut oil, corn oil, hazelnut oil, carite butter, palm oil, apricot kernel oil, and beauty-leaf oil; the oils of animal origin may be chosen from perhydrosqualene, and the oils of mineral origin may be chosen from liquid paraffin and liquid petroleum jelly.

The polyisobutenes and poly(α-olefins) useful herein may be chosen from those well known in the art.

The esters useful herein may be chosen from fatty acid esters, for example, isopropyl myristate, isopropyl palmitate, 2-ethylhexyl palmitate, purcellin oil (stearyl octanoate), isononyl isononanoate, isostearyl isononanoate and isopropyl lanolate.

The anionic polymers that may, for example, be used in the compositions disclosed herein may be chosen from polymers comprising at least one group derived from carboxylic acids, sulphonic acids and phosphoric acids, and having a weight-average molecular mass ranging from 500 to 5 000 000.

The carboxylic groups may, for example, be chosen from unsaturated monocarboxylic or dicarboxylic acid monomers such as those corresponding to the formula below:

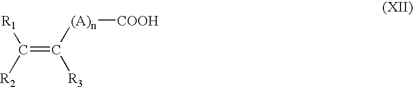

wherein:

n is an integer ranging from 0 to 10;

A is a methylene group, optionally connected to the carbon atom of the unsaturated group or to the neighboring methylene group when n is greater than 1 via a hetero atom such as oxygen or sulphur;

R₁ is chosen from a hydrogen atom and phenyl and benzyl groups;

R₂ is chosen from a hydrogen atom and lower alkyl and carboxyl groups; and

R₃ is chosen from a hydrogen atom, lower alkyl groups, a —CH₂—COOH group, and phenyl and benzyl groups.

In formula (XII) above, a lower alkyl group may, for example, comprise from 1 to 4 carbon atoms and, further, for example, may be chosen from methyl and ethyl groups.

The anionic polymers comprising at least one carboxylic group which may be used in the compositions disclosed herein may, for example, be chosen from:

A) homo- or copolymers of acrylic or methacrylic acid and salts thereof, for example, the products sold under the names Versicol® E and K by the company Allied Colloid, and Ultrahold® by the company BASF; the copolymers of acrylic acid and acrylamide sold in the form of their sodium salt under the names Reten® 421, 423 and 425 by the company Hercules; and the sodium salts of polyhydroxycarboxylic acids;

B) copolymers of acrylic or methacrylic acids with a monoethylenic monomer such as ethylene, styrene, vinyl esters and acrylic or methacrylic acid esters. These copolymers are optionally grafted onto a polyalkylene glycol such as polyethylene glycol and optionally crosslinked. Such polymers are described, for example, in French Patent No.1 222 944 and German Patent Application No. 2 330 956. For example, the copolymer may be chosen from copolymers whose chain comprises at least one optionally N-alkylated and/or hydroxyalkylated acrylamide unit, such as those described, for example, in the Luxembourg Patent Application Nos. 75370 and 75371 and sold under the name Quadramer® by the company American Cyanamid; copolymers of acrylic acid and of C₁-C₄ alkyl methacrylates; and copolymers of methacrylic acid and of ethyl acrylate sold under the name Luvimer® MAEX by the company BASF;

C) copolymers derived from crotonic acid, such as those whose chain comprises at least one unit chosen from vinyl acetate and propionate units and optionally other monomers such as allylic and methallylic esters, vinyl ether or vinyl ester of a saturated, linear or branched carboxylic acid comprising at least one long hydrocarbon-based chain such as those comprising at least 5 carbon atoms, it being possible for these polymers to be grafted and crosslinked, or alternatively a vinyl, allylic or methallylic ester of an α- or β-cyclic carboxylic acid. Such polymers are described, inter alia, in French Patent Nos. 1 222 944, 1 580 545, 2 265 782, 2 265 781, 1 564 110 and 2 439 798. Commercial products chosen from this category include the resins 28-29-30, 26-13-14 and 28-13-10 sold by the company National Starch;

D) polymers derived from maleic, fumaric or itaconic acids or anhydrides with vinyl esters, vinyl ethers, vinyl halides, phenylvinyl derivatives or acrylic acid and the esters thereof; these polymers may be esterified. Such polymers are described, for example, in U.S. Pat. Nos. 2,047,398, 2,723,248 and 2,102,113 and GB Patent No. 839 805, and, for example, those sold under the names Gantrez® AN and ES by the company ISP.

Polymers also chosen from this category include the copolymers of maleic, citraconic or itaconic anhydrides and of allylic or methallylic esters optionally comprising at least one group chosen from acrylamide and methacrylamide groups, α-olefins, acrylic and methacrylic esters, acrylic and methacrylic acid and vinylpyrrolidone in their chain, the anhydride functional groups being monoesterified or monoamidated. These polymers are described, for example, in French Patent Nos. 2 350 384 and 2 357 241; and

E) polyacrylamides comprising at least one carboxylate group.

The polymers comprising at least one sulphonic group may, for example, be chosen from polymers comprising at least one unit chosen from vinylsulphonic, styrenesulphonic, naphthalenesulphonic and acrylamidoalkylsulphonic units.

These polymers can be chosen, for example, from:

polyvinylsulphonic acid salts with a molecular weight ranging from 1000 to 100 000, as well as copolymers with at least one unsaturated comonomer chosen from, for example, acrylic and methacrylic acids and esters thereof, as well as acrylamide and derivatives thereof, vinyl ethers and vinylpyrrolidone;

polystyrenesulphonic acid salts, the sodium salts having a molecular weight ranging from 500 000 to 100 000, sold, respectively, under the names Flexan® 500 and Flexan® 130 by National Starch. These compounds are described in Patent No. FR 2 198 719;

polyacrylamidesulphonic acid salts such as those described in U.S. Pat. No. 4,128,631, for example, polyacrylamidoethylpropanesulphonic acid sold under the name Cosmedia Polymer® HSP 1180 by Henkel.

As used herein, the anionic polymers may, for example, be chosen from acrylic acid copolymers such as the acrylic acid/ethyl acrylate/N-tert-butylacrylamide terpolymer sold under the name Ultrahold Strong® by the company BASF, copolymers derived from crotonic acid, such as the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, polymers derived from maleic, fumaric or itaconic acids or anhydrides with vinyl esters, vinyl ethers, vinyl halides, phenylvinyl derivatives or acrylic acid and esters thereof, such as the methyl vinyl ether/monoesterified maleic anhydride copolymer sold under the name Gantrez® ES 425 by the company ISP, the copolymers of methacrylic acid and of methyl methacrylate sold under the name Eudragit® L by the company Rohm Pharma, the copolymer of methacrylic acid and of ethyl acrylate sold under the name Luvimer® MAEX by the company BASF, the vinyl acetate/crotonic acid copolymer sold under the name Luviset® CA 66 by the company BASF and the vinyl acetate/crotonic acid/polyethylene glycol terpolymer sold under the name Aristoflex® A by the company BASF.

As used herein, the anionic polymers may also be used in the form of a latex or pseudolatex, i.e., in the form of an aqueous dispersion of insoluble polymer particles.

The nonionic polymers that may be used in the compositions disclosed herein may, for example, be chosen from:

vinylpyrrolidone homopolymers;

copolymers of vinylpyrrolidone and vinyl acetate;

polyalkyloxazolines such as the polyethyloxazolines sold by the company Dow Chemical under the names Peox® 50 000, Peox® 200 000 and Peox® 500 000;

vinyl acetate homopolymers, such as the product sold under the name Appretan® EM by the company Hoechst, and the product sold under the name Rhodopas® A 012 by the company Rhône-Poulenc;

copolymers of vinyl acetate and acrylic ester, such as the product sold under the name Rhodopas® AD 310 by Rhône-Poulenc;

copolymers of vinyl acetate and ethylene, such as the product sold under the name Appretan® TV by the company Hoechst;

copolymers of vinyl acetate and maleic ester, for example of dibutyl maleate, such as the product sold under the name Appretan® MB Extra by the company Hoechst;

copolymers of polyethylene and maleic anhydride;

alkyl acrylate homopolymers and alkyl methacrylate homopolymers, such as the product sold under the name Micropearl® RQ 750 by the company Matsumoto and the product sold under the name Luhydran® A 848 S by the company BASF;

acrylic ester copolymers such as, for example, copolymers of alkyl acrylates and alkyl methacrylates, such as the products sold by the company Rohm & Haas under the names Primal® AC-261 K and Eudragit® NE 30 D, by the company BASF under the names Acronal® 601, Luhydran® LR 8833 or 8845, and by the company Hoechst under the names Appretan® N 9213 or N 921 2;

copolymers of acrylonitrile and a nonionic monomer chosen, for example, from butadiene and alkyl (meth)acrylates; for example, the products sold under the names Nipol® LX 531 8 by the company Nippon Zeon and those sold under the name CJ 0601 8 by the company Rohm & Haas;

polyurethanes, such as the products sold under the names Acrysol® RM 1020 and Acrysol® RM 2020 by the company Rohm & Haas, and the products Uraflex® XP 401 UZ and Uraflex® XP 402 UZ by the company DSM Resins;

copolymers of alkyl acetate and urethane, such as the product 8538-33 by the company National Starch;

polyamides, such as the product Estapor® LO 11 sold by the company Rhône-Poulenc; and

unmodified and chemically modified nonionic guar gums.

The unmodified nonionic guar gums may, for example, be chosen from the products sold under the name Vidogum® GH 175 by the company Unipectine and under the name Jaguar® C by the company Meyhall.

The modified nonionic guar gums that can be used in the compositions disclosed herein may, for example, be modified with at least one group chosen from C₁-C₆ hydroxyalkyl groups. For example, the at least one group can be chosen from hydroxymethyl, hydroxyethyl, hydroxypropyl and hydroxybutyl groups.

These guar gums are well known in the state of the art and can be prepared, for example, by reacting corresponding alkene oxides, such as, for example, propylene oxides, with guar gum so as to obtain a guar gum modified with at least one hydroxypropyl group.

Such nonionic guar gums optionally modified with at least one hydroxyalkyl groups are sold, for example, under the trade names Jaguar® HP8, Jaguar® HP60 and Jaguar® HP120, Jaguar® DC 293 and Jaguar® HP 105 by the company Meyhall and under the name Galactasol® 4H4FD2 by the company Aqualon.

The alkyl groups of the nonionic polymers may, for example, comprise from 1 to 6 carbon atoms.

The reducing agents that may be used in the compositions disclosed herein may, for example, be chosen from thio acids and salts thereof, such as thioglycolic acid, thiosulphate, cysteine, and cysteamine; alkali metal sulphites and alkaline-earth metal sulphites; reducing sugars such as glucose; vitamin C and derivatives thereof; sulphinic acid derivatives; and phosphines.

The dyes may be chosen from conjugated linear and aromatic, heterocyclic and non-heterocyclic, structures. For example, the dyes may be chosen from nitrobenzene dyes, aromatic dyes, aminobenzene dyes, azo dyes, anthraquinone dyes, aromatic diamines, aminophenols, phenols and naphthols, porphyrins, tetraphenylporphyrins, metalloporphyrins, phthalocyanins, carotenoids, flavanoids and fluorescent molecules, such as fluorescein, rhodamine, and coumarin, etc.

The at least one complexing agent (b) may, for example, be chosen from the compounds of formula:

D(A′)_{n′}(X)_{m′}(α′)_{p′} (II)

wherein:

α′ is chosen from anchoring groups allowing covalent attachment to a keratin material;

X is chosen from groups Z′ and radicals CW′;

W′, A′, m′, n′ and p′ have the same possible meanings as W, A, m, n and p, respectively, in formula (I);

Z′ is chosen from carboxyl, mercaptan, alkoxycarbonyl, amino, ammonio, phosphite, phosphate, phosphonate, sulphate sulphite, and sulphonate radicals;

C is chosen from moieties for attaching the residue W to the active complexing component D; and

D is such that the combination D(A′)_{n′}(X)_{m′}(α′)_{p′} can form, with the at least one compound (a), a complex whose dissociation constant is less than 10⁻¹ at a temperature of 25° C. D may, for example, be chosen from antibodies such as fragments of the Fab, Fv and scFv type. D may also, for example, be chosen from enzymes, a lectin, CBD (Cellulose Binding Domain), and nucleic acid monostrands; provided that that when (a) and (b) are present in the same composition, D and L do not denote complementary nucleic acid strands.

In compounds (a) or (b), the anchoring groups α and α′ may, for example, be independently chosen from the following groups: thiosulphates; aldehydes; epoxides; alkoxysilane; silanol; aziridine; acetal; hemiacetal; aminal; hemiaminal; oxazine and oxazoline; oxazinium and oxazolinium; vinyl and activated vinyl; acrylonitrile, acrylic and methacrylic esters, crotonic acid and esters, cinnamic acids and esters, styrene and derivatives thereof, butadiene, vinyl ethers, vinylacetone, maleic esters, vinyl sulphones, maleimides; ketones and α-hydroxy ketones, α-halo ketones; alkyl, aryl and arylalkyl halides RX, wherein X=I, Br or Cl; halides of unsaturated rings, such as carbon-based rings and heterocycles, RX: chlorotriazine, chloropyrimidine, chloroquinoxaline, chlorobenzotriazole; sulphonyl halide: RSO₂Cl or —F; (in the last 3 cases, group X may also be chosen from OSO₃H, N(Me)₃, SO₂Me, OPO₃H, SO₂Et groups); para-nitrophenyl ester; pentafluorophenyl ester siloxane; halogenosilanes; halogenoacetates; hydrazines; acids; difluorodinitrobenzene; N-hydroxysuccinimide esters; pyridyldithio; nitrobenzyldithio, imidate; phenyl anhydrides and azides, benzophenone, acid halides, lactones and thiolactones, isocyanate and isothiocyanate; and diazoalkanes and thiols.

The compositions disclosed herein may comprise the at least one compound (a) and the at least one complexing agent (b) described above, in a total amount ranging, for example, from 0.000001% to 90% by weight, relative to the total weight of the composition, further, for example, ranging from 0.000005% to 50% by weight, relative to the total weight of the composition, and even further, for example, ranging from 0.00001% to 20% by weight, relative to the total weight of the composition.

The compositions disclosed herein may further comprise a cosmetically acceptable medium which may be chosen, for example, from water, mixtures of solvents, and mixtures of water and at least one cosmetically acceptable solvent chosen, for example, from C₁-C₄ lower alcohols, for example, ethanol, isopropanol, tert-butanol and n-butanol; and alkylene glycols, for example, propylene glycol, and glycol ethers.

The pH of the compositions disclosed herein may range, for example, from 3 to 10 and further, for example, range from 5 to 7.

The compositions disclosed herein may also comprise at least one additive chosen from natural and synthetic, anionic, amphoteric, zwitterionic, nonionic and cationic, associative and non-associative polymeric thickeners, non-polymeric thickeners, for example, acids and electrolytes, anionic, nonionic and amphoteric surfactants, nacreous agents, opacifiers, fragrances, colorants, mineral and organic particles, preserving agents and pH stabilizers.

A person skilled in the art will take care to select the optional additive(s) and the amount thereof such that they do not harm the properties of the treatment compositions.

The at least one additive may, for example, be present in the compositions disclosed herein in an amount ranging from 0 to 20% by weight, relative to the total weight of the composition.

These compositions disclosed herein may, for example, be provided in a form chosen from more or less thickened aqueous, alcoholic and aqueous-alcoholic solutions; creams; fluid gels; sticks; and mousses. The compositions may also be packaged in aerosol form.

Disclosed herein is a set of cosmetic processes for treating a keratin material, for example, hair.

A first process comprises, in a first stage, depositing onto the keratin material by adsorption the at least one compound (a) wherein p=0 and Y is chosen from groups Z, and then, in a second stage, applying the at least one complexing agent (b) wherein p′=0 and X is chosen from groups Z′.

A second process comprises, in a first stage, depositing onto the keratin material by adsorption the at least one compound (a) wherein p=0 and Y is chosen from radicals BW and then, in a second stage, applying the at least one complexing agent (b) wherein p′=0 and X is chosen from groups Z′.

A third process comprises, in a first stage, depositing onto the keratin material by adsorption the at least one compound (a) wherein p=0 and Y is chosen from groups Z and then, in a second stage, applying the at least one complexing agent (b) wherein p′=0 and X is chosen from radicals CW′.

A fourth process comprises, in a first stage, depositing onto the keratin material by adsorption the at least one compound (a) wherein p=0 and Y is chosen from radicals BW and then, in a second stage, applying the at least one complexing agent (b) wherein p′=0 and X is chosen from radicals CW′.

A fifth process comprises, in a first stage, depositing onto the keratin material by adsorption the at least one complexing agent (b) wherein p′=0 and X is chosen from groups Z′, and then, in a second stage, applying the at least one compound (a) wherein p=0 and Y is chosen from groups Z.

A sixth process comprises, in a first stage, depositing onto the keratin material by adsorption at least one complexing agent (b) wherein p′=0 and X is chosen from groups Z′, and then, in a second stage, applying the at least one compound (a) wherein: p=0 and Y is chosen from radicals BW.

A seventh process comprises, in a first stage, depositing onto the keratin materials by adsorption at least one complexing agent (b) wherein p′=0 and X is chosen from radicals CW′, and then, in a second stage, applying the at least one compound (a) wherein p=0 and Y is chosen from groups Z.

An eighth process comprises, in a first stage, depositing onto the keratin material by adsorption at least one complexing agent (b) wherein p′=0 and X is chosen from radicals CW′, and then, in a second stage, applying the at least one compound (a) wherein p=0 and Y is chosen from radicals BW.

A ninth process comprises, in a first stage, depositing onto the keratin material by adsorption the at least one compound (a) wherein p=0 and Y is chosen from groups Z, and then, in a second stage, applying the at least one compound (b) wherein p′≠0 and X is chosen from groups Z′.

A tenth process comprises, in a first stage, depositing onto the keratin material by adsorption the at least one compound (a) wherein p=0 and Y is chosen from radicals BW, and then, in a second stage, in applying the at least one complexing agent (b) wherein p′≠0 and X is chosen from groups Z′.

An eleventh process comprises, in a first stage, depositing onto the keratin material by adsorption the at least one compound (a) wherein p=0 and Y is chosen from groups Z, and then, in a second stage, applying the at least one complexing agent (b) wherein p′≠0 and X is chosen from radicals CW′.

A twelfth process comprises, in a first stage, depositing onto the keratin material by adsorption the at least one compound (a) wherein p=0 and Y is chosen from radicals BW, and then, in a second stage, applying the at least one complexing agent b) wherein p′≠0 and X is chosen from radicals CW′.

A thirteenth process comprises, in a first stage, depositing onto the keratin material by adsorption the at least one complexing agent (b) wherein p′=0 and X is chosen from groups Z′, and then, in a second stage, applying the at least one compound a) wherein p≠0 and Y is chosen from groups Z.

A fourteenth process comprises, in a first stage, depositing onto the keratin material by adsorption the at least one complexing agent (b) wherein p′=0 and X is chosen from groups Z′, and then, in a second stage, applying the at least one compound (a) wherein p≠0 and Y is chosen from radicals BW.

A fifteenth process comprises, in a first stage, depositing onto the keratin material by adsorption the at least one complexing agent (b) wherein p′=0 and X is chosen from radicals CW′, and then, in a second stage, applying the at least one compound (a) wherein p≠0 and Y is chosen from groups Z.

A sixteenth process comprises, in a first stage, depositing onto the keratin material by adsorption the at least one complexing agent (b) wherein p′=0 and X is chosen from radicals CW′, and then, in a second stage, in applying the at least one compound (a) wherein p≠0 and Y is chosen from radicals BW.

A seventeenth process comprises, in a first stage, depositing onto the keratin material by covalent attachment the at least one compound (a) wherein p≠0 and Y is chosen from groups Z, and then, in a second stage, applying the at least one complexing agent (b) wherein p′=0 and X is chosen from groups Z′.

An eighteenth process comprises, in a first stage, depositing onto the keratin material by covalent attachment the at least one compound (a) wherein p≠0 and Y is chosen from radicals BW, and then, in a second stage, applying the at least one complexing agent (b) wherein p′=0 and X is chosen from groups Z′.

A nineteenth process comprises, in a first stage, depositing onto the keratin material by covalent attachment the at least one compound (a) wherein p≠0 and Y is chosen from groups Z, and then, in a second stage, applying the at least one complexing agent (b) wherein p′=0 and X is chosen from radicals CW′.

A twentieth process comprises, in a first stage, depositing onto the keratin material by covalent attachment the at least one compound (a) wherein p≠0 and Y is chosen from radicals BW, and then, in a second stage, applying the at least one complexing agent (b) wherein p′=0 and X is chosen from radicals CW′.

A twenty-first process comprises, in a first stage, depositing onto the keratin material by covalent attachment the at least one complexing agent (b) wherein p′≠0 and X is chosen from groups Z′, and then, in a second stage, applying the at least one compound (a) wherein p=0 and Y is chosen from groups Z.

A twenty-second process comprises, in a first stage, depositing onto the keratin material by covalent attachment the at least one complexing agent b) wherein p′≠0 and X is chosen from groups Z′, and then, in a second stage, applying the at least one compound (a) wherein p=0 and Y is chosen from radicals BW.

A twenty-third process comprises, in a first stage, depositing onto the keratin material by covalent attachment the at least one complexing agent (b) wherein p′≠0 and X is chosen from radicals CW′, and then, in a second stage, applying the at least one compound (a) wherein p=0 and Y is chosen from groups Z.

A twenty-fourth process comprises, in a first stage, depositing onto the keratin material by covalent attachment the at least one complexing agent (b) wherein p′≠0 and X is chosen from radicals CW′, and then, in a second stage, applying the at least one compound (a) wherein p=0 and Y is chosen from radicals BW.

A twenty-fifth process comprises, in a first stage, depositing onto the keratin material by covalent attachment the at least one compound (a) wherein p≠0 and Y is chosen from groups Z, and then, in a second stage, applying the at least one complexing agent (b) wherein p′≠0 and X is chosen from groups Z′.

A twenty-sixth process comprises, in a first stage, depositing onto the keratin material by covalent attachment the at least one compound (a) wherein p≠0 and Y is chosen from radicals BW, and then, in a second stage, applying the at least one complexing agent (b) wherein p′≠0 and X is chosen from groups Z′.

A twenty-seventh process comprises, in a first stage, depositing onto the keratin material by covalent attachment the at least one compound (a) wherein p≠0 and Y is chosen from groups Z, and then, in a second stage, applying the at least one complexing agent (b) wherein p′≠0 and X is chosen from radicals CW′.

A twenty-eighth process comprises, in a first stage, depositing onto the keratin material by covalent attachment the at least one compound (a) wherein p≠0 and Y is chosen from radicals BW, and then, in a second stage, applying the at least one complexing agent (b) wherein p′≠0 and X is chosen from radicals CW′.

A twenty-ninth process comprises, in a first stage, depositing onto the keratin material by covalent attachment the at least one complexing agent (b) wherein p′≠0 and X is chosen from groups Z′, and then, in a second stage, applying the at least one compound (a) wherein p≠0 and Y is chosen from groups Z.

A thirtieth process comprises, in a first stage, depositing onto the keratin material by covalent attachment the at least one complexing agent b) wherein p′≠0 and X is chosen from groups Z′, and then, in a second stage, applying the at least one compound (a) wherein p≠0 and Y is chosen from radicals BW.

A thirty-first process comprises, in a first stage, depositing onto the keratin material by covalent attachment at least one complexing agent (b) wherein p′≠0 and X is chosen from radicals CW′, and then, in a second stage, applying the at least one compound a) wherein p≠0 and Y is chosen from groups Z.

A thirty-second process comprises, in a first stage, depositing onto the keratin material by covalent attachment the at least one complexing agent (b) wherein p′≠0 and X is chosen from radicals CW′, and then, in a second stage, applying the at least one compound (a) wherein p≠0 and Y is chosen from radicals BW.

A thirty-third process comprises simultaneously depositing onto the keratin material by absorption the at least one compound (a) wherein p=0 and Y is chosen from groups Z and the at least one complexing agent (b) wherein p′=0 and X is chosen from groups Z′.

A thirty-fourth process comprises simultaneously depositing onto the keratin material by absorption the at least one compound (a) wherein p=0 and Y is chosen from radicals BW and the at least one complexing agent (b) wherein p′=0 and X is chosen from groups Z′.

A thirty-fifth process comprises simultaneously depositing onto the keratin material by absorption the at least one compound (a) wherein p=0 and Y is chosen from groups Z and the at least one complexing agent (b) wherein p′=0 and X is chosen from radicals CW′.

A thirty-sixth process comprises simultaneously depositing onto the keratin material by absorption the at least one compound (a) wherein p=0 and Y is chosen from radicals BW and the at least one complexing agent b) wherein p′=0 and X is chosen from radicals CW′.

A thirty-seventh process comprises simultaneously depositing onto the keratin material the at least one compound (a) wherein p=0 and Y is chosen from groups Z and the at least one complexing agent (b) wherein p′≠0 and X is chosen from groups Z′.

A thirty-eighth process comprises simultaneously depositing onto the keratin material the at least one compound (a) wherein p=0 and Y is chosen from radicals BW and the at least one complexing agent (b) wherein p′≠0 and X is chosen from groups Z′.

A thirty-ninth process comprises simultaneously depositing onto the keratin material the at least one compound (a) wherein p=0 and Y is chosen from groups Z and the at least one complexing agent (b) wherein p′≠0 and X is chosen from radicals CW′.

A fortieth process comprises simultaneously depositing onto the keratin material at least one compound (a) wherein p=0 and Y is chosen from radicals BW and the at least one complexing agent (b) wherein p′≠0 and X is chosen from radicals CW′.

A forty-first process comprises simultaneously depositing onto the keratin material the at least one compound a) wherein p≠0 and Y is chosen from groups Z and at least one complexing agent (b) wherein p′=0 and X is chosen from groups Z′.

A forty-second process comprising simultaneously depositing onto the keratin material the at least one compound (a) wherein p≠0 and Y is chosen from radicals BW and the at least one complexing agent (b) wherein p′=0 and X is chosen from groups Z′.

A forty-third process comprises simultaneously depositing onto the keratin material at least one compound (a) wherein p≠0 and Y is chosen from groups Z and the at least one complexing agent (b) wherein p′=0 and X is chosen from radicals CW′.

A forty-fourth process comprises simultaneously depositing onto the keratin material the at least one compound (a) wherein p≠0 and Y is chosen from radicals BW and the at least one complexing agent (b) wherein p′=0 and X is chosen from radicals CW′.

A forty-fifth process comprises simultaneously depositing onto the keratin material the at least one compound (a) wherein p≠0 and Y is chosen from groups Z and the at least one complexing agent (b) wherein p′≠0 and X is chosen from groups Z′.

A forty-sixth process comprises simultaneously depositing onto the keratin material the at least one compound (a) wherein p≠0 and Y is chosen from radicals BW and the at least one complexing agent (b) wherein p′≠0 and X is chosen from groups Z′.

A forty-seventh process comprises simultaneously depositing onto the keratin material the at least one compound (a) wherein p≠0 and Y is chosen from groups Z and the at least one complexing agent (b) wherein p′≠0 and X is chosen from radicals CW′.

A forth-eighth process comprises simultaneously depositing onto the keratin materials the at least one compound (a) wherein p≠0 and Y is chosen from radicals BW and the at least one complexing agent (b) wherein p′≠0 and X is chosen from radicals CW′.

Optionally, for all the processes, it is possible to introduce the at least one compound a) or the at least one complexing agent b) in at least one additional step. Multilayer deposits may thus be formed.

Further disclosed herein are multi-compartment kits.

A first kit comprises, in a first compartment, the at least one compound (a) wherein p=0 and Y is chosen from groups Z and, in a second compartment, the at least one complexing agent (b) wherein p′=0 and X is chosen from groups Z′.

A second kit comprises, in a first compartment, the at least one compound a) wherein p=0 and Y is chosen from radicals BW and, in a second compartment, the at least one complexing agent (b) wherein p′=0 and X is chosen from groups Z′.

A third kit comprises, in a first compartment, the at least one compound (a) wherein p=0 and Y is chosen from groups Z and, in a second compartment, the at least one complexing agent (b) wherein p′=0 and X is chosen from radicals CW′.

A fourth kit comprises, in a first compartment, the at least one compound (a) wherein p=0 and Y is chosen from radicals BW and, in a second compartment, the at least one complexing agent (b) wherein p′=0 and X is chosen from radicals CW′.

A fifth kit comprises, in a first compartment, the at least one compound (a) wherein p=0 and Y is chosen from groups Z and, in a second compartment, the at least one complexing agent (b) wherein p′≠0 and X is chosen from groups Z′.

A sixth kit comprises, in a first compartment, the at least one compound (a) wherein p=0 and Y is chosen from radicals BW and, in a second compartment, the at least one complexing agent (b) wherein p′≠0 and X is chosen from groups Z′.

A seventh kit comprises, in a first compartment, the at least one compound (a) wherein p=0 and Y is chosen from groups Z and, in a second compartment, the at least one complexing agent (b) wherein p′≠0 and X is chosen from radicals CW′.

An eighth kit comprises, in a first compartment, the at least one compound (a) wherein p=0 and Y is chosen from radicals BW and, in a second compartment, the at least one complexing agent b) wherein p′≠0 and X is chosen from radicals CW′.

A ninth kit comprises, in a first compartment, the at least one compound (a) wherein p≠0 and Y is chosen from groups Z and, in a second compartment, the at least one complexing agent (b) wherein p′=0 and X is chosen from groups Z′.

A tenth kit comprises, in a first compartment, the at least one compound (a) wherein p≠0 and Y is chosen from radicals BW and, in a second compartment, the at least one complexing agent (b) wherein p′=0 and X is chosen from groups Z′.

An eleventh kit comprises, in a first compartment, the at least one compound (a) wherein p≠0 and Y is chosen from groups Z and, in a second compartment, the at least one complexing agent (b) wherein p′=0 and X is chosen from radicals CW′.

A twelfth kit comprises, in a first compartment, the at least one compound (a) wherein p≠0 and Y is chosen from radicals BW and, in a second compartment, the at least one complexing agent (b) wherein p′=0 and X is chosen from radicals CW′.

A thirteenth kit comprises, in a first compartment, the at least one compound (a) wherein p≠0 and Y is chosen from groups Z and, in a second compartment, the at least one complexing agent (b) wherein p′≠0 and X is chosen from groups Z′.

A fourteenth kit comprises, in a first compartment, the at least one compound (a) wherein p≠0 and Y is chosen from radicals BW and, in a second compartment, the at least one complexing agent b) wherein p′≠0 and X is chosen from groups Z′.

A fifteenth kit comprises, in a first compartment, the at least one compound (a) wherein p≠0 and Y is chosen from groups Z and, in a second compartment, the at least one complexing agent (b) wherein p′≠0 and X is chosen from radicals CW′.

A sixteenth kit comprises, in a first compartment, the at least one compound (a) wherein p≠0 and Y is chosen from radicals BW and, in a second compartment, the at least one complexing agent (b) wherein p′≠0 and X is chosen from radicals CW′.

Kits comprising 3, 4, 5 or 6 compartments may also be envisaged if it is desired to form multilayer deposits.

The ingredients disclosed herein may also be introduced within existing hair treatment formulations.

The hair treatment process disclosed herein may comprise a preliminary step that comprises applying at least one cosmetic treatment agent chosen from reducing agents; oxidizing agents for permanently reshaping the hair; oxidation dyes; bleaching agents; shampoos; and styling compositions.

The examples that follow are intended to illustrate the invention without, however, being limiting in nature.

### EXAMPLE 1

### Shampoo-Fastness of an Anti-FITC-Dextran Complex

In this example, Anti-FITC (anti-fluorescein isothiocyanate) was adsorbed onto the hair and the FITC-dextran complex was then attached to the hair.

a) Adsorption of Anti-FITC onto the Surface of the Hair

5 hairs (about 10 mg) derived from a lock of washed natural hair were taken for each sample and placed in silanized glass flasks.

A solution of Anti-FITC (Sigma F-5636) at 50 μg/ml in a PBS buffer (0.1M, pH 7.2) was prepared. 800 μl of this Anti-FITC solution were placed in contact with the hair sample. The mixture was stirred for 24 hours at room temperature.

The sample was rinsed once with 1 ml of a solution of Tween 20 at 0.05% in PBS, followed by rinsing once with 1 ml of PBS and rinsing 3 times with distilled water (3×1 ml of distilled water with vortex-stirring for 1 minute).

b) Adsorption and Fastness of an FITC-Dextran Complex on the Surface of Treated Hair

A solution of FITC-dextran (Sigma, ref: FD-70S) at 100 μg/ml in phosphate buffer (pH 7.2) was prepared. Untreated hair (control) and hair treated with Anti-FITC were incubated in 1 ml of this solution for 2 hours at room temperature. The samples were then rinsed 3 times with distilled water (3×1 ml of distilled water with vortex-stirring for 1 minute).

After shampooing, the treated and control hairs were analyzed by scanning electron microscopy (SCM). The SCM images showed the presence of a heterogeneous deposit of dextran on the treated hair, whereas no dextran was detected on the control hair. These results show that the attachment and shampoo-fastness of the FITC-dextran complex on the hair was due to the FITC/Anti-FITC combination.

### EXAMPLE 2

### Shampoo-Fastness of an Anti-FITC-Biotin Complex

In this example, the Anti-FITC was adsorbed onto the hair and the FITC-Biotin complex was then attached to the hair.

a) Adsorption of Anti-FITC onto the Surface of the Hair

(see Example 1)

b) Adsorption and Fastness of an FITC-Biotin Complex on the Surface of Treated Hair

A solution of FITC-Biotin (Molecular Probes, ref: B-1370) at 100 μg/ml in phosphate buffer (pH 7.2) was prepared. Untreated hair and hair treated with Anti-FITC were incubated in 1 ml of this solution for 2 hours at room temperature.

The samples were rinsed 3 times with distilled water (with 3×1 ml of distilled water, vortex-stirring for 1 minute). The controls prepared in parallel corresponded to hair treated with Anti-FITC but not placed in contact with the FITC-Biotin.

A solution of avidin-peroxidase (Sigma, ref: A3151) at 50 μg/ml in phosphate buffer (pH 7.2) was prepared. The samples were incubated in 1 ml of this solution for 1 hour at room temperature and were then rinsed in several steps: 3 rinses with 1 ml of a shampoo solution and then 3 rinses with 1 ml of distilled water. 1 ml of a solution of peroxidase substrate (Sigma, ref: A3219) was then added. This substrate, which was colorless in solution, was converted into a colored (yellow) substance after reaction with peroxidase. This substance absorbed UV light (405 nm) strongly and thus made it possible to detect the presence of peroxidase on the hair. The reaction was left to proceed for 5 minutes and was then quenched with 1 ml of 1% SDS solution. 200 μl of the supernatant of each sample were taken up and placed, after dilution, in the wells of a 96-well plate. A spectrophotometric reading at 405 nm was taken.

The mean optical density of the solution obtained for the treated hair was 1.36. The mean optical density of the solution obtained for the control hair was 0.88.

These results showed that the better attachment and shampoo-fastness of the FITC-Biotin complex to the hair was due to the FITC/Anti-FITC combination.

### EXAMPLE 3

### Shampoo-Fastness of an Anti-FITC-Pigment Complex

In this example, PEI-FITC (polyethyleneimine-FITC) was adsorbed onto the hair and the Anti-FITC-pigment complex was then attached to the hair.

a) Preparation of the PEI-FITC Solution

500 μl of FITC (Pierce 46110) at 100 mg/ml in DMF were prepared. 400 82 l of the FITC solution were gradually added to a small beaker containing 2 ml of 5% PEI (pH 8.5). The mixture was stirred for 2 hours at room temperature while being protected from light with aluminium foil, and was then purified on a PD10 column (Amersham 17-0851-01) (eluent: 3.5 ml of distilled water).

b) Adsorption of Anti-FITC onto the Surface of the Pigments

100 mg of ochre pigment (iron oxide) was washed with 1 ml of pH 7.2 PBS. 1 ml of a solution of Anti-FITC at 500 μg/ml in PBS at pH 7.2 was added to the pigment. The mixture was stirred for 2 hours at room temperature and then centrifuged. The pigments—Anti-FITC were washed 6 times with 1 ml of pH 7.2 PBS and then suspended in 1 ml of pH 7.2 PBS.

c) Adsorption of PEI-FITC onto the Surface of the Hair

0.25 g of PEI-FITC was deposited on 1 g of grey hair. The hair was massaged and then washed with water and dried at 60° C. for 30 minutes.

d) Adsorption and Fastness of an Anti-FITC-Pigment Complex on the Surface of Treated Hair

1 ml of a pigment-anti-FITC solution and 3 ml of pH 7.2 PBS were added to 1 g of hair coated with PEI-FITC. After incubation for 2 hours at room temperature, the hair was washed with shampoo.

The grafting of pigment to the grey hair was quantified by the L.a.b. method. The measurements were performed using a Minolta CM2002 spectrophotometer, in the CI 1976 system. They were performed by reflection on the locks after drying. The tone strengths were likened to the lightness, L* (CIELAB system).

The measurements were performed on locks before and after shampooing, treated with PEI alone (control) or with PEI-FITC and then placed in contact with functionalized pigments.

These results showed that the better attachment and shampoo-fastness of the Anti-FITC-pigment complex on the hair was due to the FITC/Anti-FITC combination.

What is claimed is:

1. A cosmetic composition comprising:
at least one compound (a); and
at least one complexing agent (b) capable of forming a complex, having a dissociation constant of less than or equal to 0.1 at a temperature of 25° C., with the at least one compound (a), wherein the at least one compound (a) is chosen from compounds of formula (I):
L—(A)ₙ—(Y)ₘ—(α)ₚ (I)
wherein:
m is a number ranging from 0 to 100;
n is equal to 0 or 1;
p is a number ranging from 0 to 100;
α is chosen from anchoring groups allowing covalent attachment to a keratin material;
L is chosen from residues having complexing activity relative to the at least one complexing agent (b), provided that L is not biotin;
A is chosen from linear and branched, saturated and unsaturated divalent radicals comprising from 1 to 100 carbon atoms, optionally interrupted with at least one hetero atom and optionally substituted with at least one group chosen from hydroxyl, amino, halogen, aryl, phosphate, phosphonate, sulphate, sulphonate, carboxyl, alkoxycarbonyl and alkoxy groups;
Y is chosen from groups Z, wherein Z is chosen from carboxyl, mercaptan, alkoxycarbonyl, amino, ammonio, phosphite, phosphate, phosphonate, sulphate sulphite, and sulphonate and from radicals BW, wherein W is chosen from residues with cosmetic activity and B is chosen from species for fixing the residue W to L or to A;
B is chosen from amine, imine, amide, ester, disulphide, thioester, urethane, urea, ether, thioether, azo, methine and epoxy groups, aromatic and heterocyclic residues and dialkylpolysiloxane residues; and
W is derived from at least one of UV-screening molecules, hydrating and emollient molecules, conditioners, antistatic agents, antiperspirants, fragrancing materials, reducing molecules, oxidizing molecules, coloring molecules, antimicrobial agents, antidandruff agents, mineral and organic particulates onto which are optionally adsorbed at least one polymer, and anionic, nonionic, amphoteric and cationic film-forming agents onto which are optionally adsorbed organic and/or mineral particles.

2. The composition according to claim 1, wherein the at least one complexing agent (b) is chosen from compounds of formula (II):
D(A′)_{n′}(X)_{m′}(α′)_{p′} (II)
wherein:
α′ is chosen from anchoring groups allowing covalent attachment to a keratin material;
X is chosen from groups Z′ and radicals CW′;
Z′ is chosen from carboxyl, mercaptan, alkoxycarbonyl, amino, ammonio, phosphite, phosphate, phosphonate, sulphate sulphite, and sulphonate radicals;
m′ is a number ranging from 0 to 100;
n′ is equal to 0 or 1;
p′ is a number ranging from 0 to 100;
A′ is chosen from linear and branched, saturated and unsaturated divalent radicals comprising from 1 to 100 carbon atoms, optionally interrupted with at least one hetero atom and optionally substituted with at least one group chosen from hydroxyl, amino, halogen, aryl, phosphate, phosphonate, sulphate, sulphonate, carboxyl, alkoxycarbonyl and alkoxy groups;
W′ is derived from at least one of UV-screening molecules, hydrating and emollient molecules, conditioners, antistatic agents, antiperspirants, fragrancing materials, reducing molecules, oxidizing molecules, coloring molecules, antimicrobial agents, antidandruff agents, mineral and organic particulates onto which are optionally adsorbed at least one polymer, and anionic, nonionic, amphoteric and cationic film-forming agents onto which are optionally adsorbed organic and/or mineral particles;
C is chosen from moieties for attaching the residue W to the active complexing component D; and
D is such that the combination D(A′)_{n′}(X)_{m′}(α′)_{p′} can form with the at least one compound (a) a complex whose dissociation constant is less than 0.1 at a temperature of 25° C.

3. The cosmetic composition according to claim 1, wherein, in the at least one compound (a), the anchoring group a is chosen from: thiosulphates; aldehydes; epoxides; alkoxysilane; silanol; aziridine; acetal; hemiacetal; aminal; hemiaminal; oxazine and oxazoline; oxazinium and oxazolinium; vinyl and activated vinyl; acrylonitrile, acrylic and methacrylic esters, crotonic acid and esters, cinnamic acids and esters, styrene and derivatives thereof, butadiene, vinyl ethers, vinylacetone, maleic esters, vinyl sulphones, maleimides; ketones and α-hydroxy ketones, α-halo ketones; alkyl, aryl and arylalkyl halides RX, wherein X is chosen from I, Br and Cl atoms and OSO₃H, N(Me)₃, SO₂Me, OPO₃H, and SO₂Et groups; halides RX of unsaturated rings chosen from carbon-based rings and heterocycles, wherein X is chosen from Cl, OSO₃H, N(Me)₃, SO₂Me, OPO₃H, and SO₂Et groups; sulphonyl halides RSO₂X, wherein X is chosen from Cl and F atoms and OSO₃H, N(Me)₃, SO₂Me, OPO₃H, and SO₂Et groups; para-nitrophenyl esters; pentafluorophenyl ester siloxane; halogenosilanes; halogenoacetates; hydrazines; acids; difluorodinitrobenzene; N-hydroxysuccinimide esters; pyridyldithio; nitrobenzyldithio, imidate; phenyl anhydrides or azide, benzophenone, acid halides, lactones and thiolactones, isocyanate and isothiocyanate; diazoalkanes and thiols.

4. The cosmetic composition according to claim 3, wherein the heterocycles RX are chosen from chlorotriazine, chloropyrimidine, chloroquinoxaline, and chlorobenzotriazole.

5. The cosmetic composition according to claim 2, wherein, in the at least one complexing agent (b), the anchoring group α′ is chosen from: thiosulphates; aldehydes; epoxides; alkoxysilane; silanol; aziridine; acetal; hemiacetal; aminal; hemiaminal; oxazine and oxazoline; oxazinium and oxazolinium; vinyl and activated vinyl; acrylonitrile, acrylic and methacrylic esters, crotonic acid and esters, cinnamic acids and esters, styrene and derivatives thereof, butadiene, vinyl ethers, vinylacetone, maleic esters, vinyl sulphones, maleimides; ketones and α-hydroxy ketones, α-halo ketones; alkyl, aryl and arylalkyl halides RX, wherein X is chosen from I, Br and Cl atoms and OSO₃H, N(Me)₃, SO₂Me, OPO₃H, and SO₂Et groups; halides RX of unsaturated rings chosen from carbon-based rings and heterocycles, wherein X is chosen from Cl and OSO₃H, N(Me)₃, SO₂Me, OPO₃H, and SO₂Et groups; sulphonyl halides RSO₂X, wherein X is chosen from Cl and F atoms and OSO₃H, N(Me)₃, SO₂Me, OPO₃H, and SO₂Et groups; para-nitrophenyl esters; pentafluorophenyl ester siloxane; halogenosilanes; halogenoacetates; hydrazines; acids; difluorodinitrobenzene; N-hydroxysuccinimide esters; pyridyldithio; nitrobenzyldithio, imidate; phenyl anhydrides or azide, benzophenone, acid halides, lactones and thiolactones, isocyanate and isothiocyanate; diazoalkanes and thiols.

6. The cosmetic composition according to claim 5, wherein the heterocycles RX are chosen from chlorotriazine, chloropyrimidine, chloroquinoxaline, and chlorobenzotriazole.

7. A composition comprising at least one compound (a) chosen from compounds of formula (I):
L—(A)ₙ—(Y)ₘ—(α)ₚ (I)
wherein:
m is a number ranging from 0 to 100;
n is equal to 0 or 1;
p is a number ranging from 0 to 100;
α is chosen from anchoring groups allowing covalent attachment to a keratin material;
L is chosen from residues having complexing activity relative to the at least one complexing agent (b), provided that L is not biotin;
A is chosen from linear and branched, saturated and unsaturated divalent radicals comprising from 1 to 100 carbon atoms, optionally interrupted with at least one hetero atom and optionally substituted with at least one group chosen from hydroxyl, amino, halogen, aryl, phosphate, phosphonate, sulphate, sulphonate, carboxyl, alkoxycarbonyl and alkoxy groups;
Y is chosen from groups Z, wherein Z is chosen from carboxyl, mercaptan, alkoxycarbonyl, amino, ammonio, phosphite, phosphate, phosphonate, sulphate sulphite, and sulphonate and radicals BW, wherein W is chosen from residues with cosmetic activity and B is chosen from species for fixing the residue W to L or to A;
B is chosen from amine, imine, amide, ester, disulphide, thioester, urethane, urea, ether, thioether, azo, methine and epoxy groups, aromatic and heterocyclic residues and dialkylpolysiloxane residues; and
W is derived from at least one of UV-screening molecules, hydrating and emollient molecules, conditioners, antistatic agents, antiperspirants, fragrancing materials, reducing molecules, oxidizing molecules, coloring molecules, antimicrobial agents, antidandruff agents, mineral and organic particulates onto which are optionally adsorbed at least one polymer, and anionic and nonionic, amphoteric and cationic film-forming agents onto which are optionally adsorbed organic and/or mineral particles.

8. A composition comprising at least one complexing compound (b) chosen from compounds of formula (II):
D(A′)_{n′}(X)_{m′}(α′)_{p′} (II)
wherein:
α′ is chosen from anchoring groups allowing covalent attachment to a keratin material;
X is chosen from groups Z′ and radicals CW′;
Z′ is chosen from carboxyl, mercaptan, alkoxycarbonyl, amino, ammonio, phosphite, phosphate, phosphonate, sulphate sulphite, and sulphonate radicals;
m′ is a number ranging from 0 to 100;
n′ is equal to 0 or 1;
p′ is a number ranging from 0 to 100;
A′ is chosen from linear and branched, saturated and unsaturated divalent radicals comprising from 1 to 100 carbon atoms, optionally interrupted with at least one hetero atom and optionally substituted with at least one group chosen from hydroxyl, amino, halogen, aryl, phosphate, phosphonate, sulphate, sulphonate, carboxyl, alkoxycarbonyl and alkoxy groups;
C is chosen from moieties for attaching the residue W to the active complexing component D; and
D is such that the combination D(A′)_{n′}(X)_{m′}(α′)_{p′} can form with the at least one compound (a) a complex whose dissociation constant is less than 0.1 at a temperature of 25° C.

9. The composition according to claim 1, wherein the at least one compound (a) and the at least one complexing agent (b) are, respectively, derived from an antigen and an antibody.

10. The composition according to claim 1, wherein the at least one compound (a) and the at least one complexing agent (b) are derived, respectively, from carbohydrate and lectin.

11. The composition according to claim 1, wherein the at least one compound (a) and the at least one complexing agent b) are derived, respectively, from a substrate and an enzyme.

12. The composition according to claim 1, wherein the at least one compound (a) and the at least one complexing agent (b) are present in the composition in a combined amount ranging from 0.000001% to 90% by weight, relative to the total weight of the composition.

13. The composition according to claim 12, wherein the at least one compound (a) and the at least one complexing agent (b) are present in the composition in a combined amount ranging from 0.000005% to 50% by weight, relative to the total weight of the composition.

14. The composition according to claim 13, wherein the at least compound (a) and the at least one complexing agent (b) are present in the composition in a combined amount ranging from 0.00001% to 20% by weight, relative to the total weight of the composition.

15. The composition according to claim 1, wherein the composition is provided in a form chosen from at least partially thickened aqueous, alcoholic and aqueous-alcoholic solutions, creams, fluid gels, sticks, mousses, and optionally packaged in aerosol forms.

16. A process for treating a keratin material comprising:
depositing on to the keratin material at least one compound (a) chosen from compounds of formula (I):
L—(A)ₙ—(Y)ₘ—(α)ₚ (I)
m is a number ranging from 0 to 100;
n is equal to 0 or 1;
p is a number ranging from 0 to 100;
α is chosen from anchoring groups allowing covalent attachment to a keratin material;
L is chosen from residues having complexing activity relative to the at least one complexing agent (b), provided that L is not biotin;
A is chosen from linear and branched, saturated and unsaturated divalent radicals comprising from 1 to 100 carbon atoms, optionally interrupted with at least one hetero atom and optionally substituted with at least one group chosen from hydroxyl, amino, halogen, aryl, phosphate, phosphonate, sulphate, sulphonate, carboxyl, alkoxycarbonyl and alkoxy groups;
Y is chosen from groups Z, wherein Z is chosen from carboxyl, mercaptan, alkoxycarbonyl, amino, ammonio, phosphite, phosphate, phosphonate, sulphate sulphite, and sulphonate and radicals BW, wherein W is chosen from residues with cosmetic activity and B is chosen from species for fixing the residue W to L or to A;
B is chosen from amine, imine, amide, ester, disulphide, thioester, urethane, urea, ether, thioether, azo, methine and epoxy groups, aromatic and heterocyclic residues and dialkylpolysiloxane residues; and
W is derived from at least one of UV-screening molecules, hydrating and emollient molecules, conditioners, antistatic agents, antiperspirants, fragrancing materials, reducing molecules, oxidizing molecules, coloring molecules, antimicrobial agents, antidandruff agents, mineral and organic particulates onto which are optionally adsorbed at least one polymer, and anionic and nonionic, amphoteric and cationic film-forming agents onto which are optionally adsorbed organic and/or mineral particles; and
applying to the keratin material at least one complexing agent (b) chosen from compounds of formula (II):
D(A′)_{n′}(X)ₘ′(α′)_{p′} (II)
wherein:
α′ is chosen from anchoring groups allowing covalent attachment to a keratin material;
X is chosen from groups Z′ and radicals CW′;
Z′ is chosen from carboxyl, mercaptan, alkoxycarbonyl, amino, ammonio, phosphite, phosphate, phosphonate, sulphate sulphite, and sulphonate radicals;
m′ is a number ranging from 0 to 100;
n′ is equal to 0 or 1;
p′ is a number ranging from 0 to 100;
A′ is chosen from linear and branched, saturated and unsaturated divalent radicals comprising from 1 to 100 carbon atoms, optionally interrupted with at least one hetero atom and optionally substituted with at least one group chosen from hydroxyl, amino, halogen, aryl, phosphate, phosphonate, sulphate, sulphonate, carboxyl, alkoxycarbonyl and alkoxy groups;
W′ is derived from at least one of UV-screening molecules, hydrating and emollient molecules, conditioners, antistatic agents, antiperspirants, fragrancing materials, reducing molecules, oxidizing molecules, coloring molecules, antimicrobial agents, antidandruff agents, mineral and organic particulates onto which are optionally adsorbed at least one polymer, and anionic and nonionic, amphoteric and cationic film-forming agents onto which are optionally adsorbed organic and/or mineral particles;
C is chosen from moieties for attaching the residue W to the active complexing component D; and
D is such that the combination D(A′)_{n′}(X)_{m′}(α′)_{p′} can form with the at least one compound (a) a complex whose dissociation constant is less than 0.1 at a temperature of 25° C.

17. The process according to claim 16, wherein the keratin material is hair.

18. The process for treating a keratin material according to claim 16, comprising, in a first stage, depositing onto the keratin material by adsorption the at least one compound (a) wherein p=0 and Y is chosen from groups Z, and then, in a second stage, applying to the keratin material the at least one complexing agent (b) wherein p′=0 and X is chosen from groups Z′.

19. The process for treating a keratin material according to claim 16, comprising, in a first stage, depositing onto the keratin material by adsorption the at least one compound (a) wherein p=0 and Y is chosen from radicals BW, and then, in a second stage, applying to the keratin material the at least one complexing agent (b) wherein p′=0 and X is chosen from groups Z′.

20. The process for treating a keratin material according to claim 16, comprising, in a first stage, depositing onto the keratin material by adsorption the at least one compound (a) wherein p=0 and Y is chosen from groups Z, and then, in a second stage, applying to the keratin material the at least one complexing agent (b) wherein p′=0 and X is chosen from radicals CW′.

21. The process for treating a keratin material according to claim 16, comprising, in a first stage, depositing onto the keratin material by adsorption the at least one compound (a) wherein p=0 and Y is chosen from radicals BW, and then, in a second stage, applying to the keratin material the at least one complexing agent (b) wherein p′=0 and X is chosen from radicals CW′.

22. The process for treating a keratin material according to claim 16, comprising, in a first stage, depositing onto the keratin material by adsorption the at least one complexing agent (b) wherein p′=0 and X is chosen from groups Z′, and then, in a second stage, applying to the keratin material the at least one compound (a) wherein p=0 and Y is chosen from groups Z.

23. The process for treating a keratin material according to claim 16, comprising, in a first stage, depositing onto the keratin material by adsorption the at least one complexing agent (b) wherein p′=0 and X is chosen from groups Z′, and then, in a second stage, applying to the keratin material the at least one compound (a) wherein p=0 and Y is chosen from radicals BW.

24. The process for treating a keratin material according to claim 16, comprising, in a first stage, depositing onto the keratin material by adsorption the at least one complexing agent (b) wherein p′=0 and X is chosen from radicals CW′, and then, in a second stage, applying to the keratin material the at least one compound (a) wherein p=0 and Y is chosen from groups Z.

25. The process for treating a keratin material according to claim 16, comprising, in a first stage, depositing onto the keratin material by adsorption the at least one complexing agent (b) wherein p′=0 and X is chosen from radicals CW′, and then, in a second stage, applying to the keratin material the at least one compound (a) wherein p=0 and Y is chosen from radicals BW.

26. The process for treating a keratin material according to claim 16, comprising, in a first stage, depositing onto the keratin material by adsorption the at least one compound (a) wherein p=0 and Y is chosen from groups Z, and then, in a second stage, applying to the keratin material the at least one complexing agent (b) wherein p′≠0 and X is chosen from groups Z′.

27. The process for treating a keratin material according to claim 16, comprising, in a first stage, depositing onto the keratin material by adsorption the at least one compound (a) wherein p=0 and Y is chosen from radicals BW, and then, in a second stage, applying to the keratin material the at least one complexing agent (b) wherein p′≠0 and X is chosen from groups Z′.

28. The process for treating a keratin material according to claim 16, comprising, in a first stage, depositing onto the keratin material by adsorption the at least one compound (a) wherein p=0 and Y is chosen from groups Z, and then, in a second stage, applying to the keratin material the at least one complexing agent (b) wherein p′≠0 and X is chosen from radicals CW′.

29. The process for treating a keratin material according to claim 16, comprising, in a first stage, depositing onto the keratin material by adsorption the at least one compound (a) wherein p=0 and Y is chosen from radicals BW, and then, in a second stage, applying to the keratin material the at least one complexing agent (b) wherein p′≠0 and X is chosen from radicals CW′.

30. The process for treating a keratin material according to claim 16, comprising, in a first stage, depositing onto the keratin material by adsorption the at least one complexing agent (b) wherein p′=0 and X is chosen from groups Z′, and then, in a second stage, applying to the keratin material the at least one compound (a) wherein p≠0 and Y is chosen from groups Z.

31. The process for treating a keratin material according to claim 16, comprising, in a first stage, depositing onto the keratin material by adsorption the at least one complexing agent (b) wherein p′=0 and X is chosen from groups Z′, and then, in a second stage, applying to the keratin material the at least one compound (a) wherein p≠0 and Y is chosen from radicals BW.

32. The process for treating a keratin material according to claim 16, comprising, in a first stage, depositing onto the keratin material by adsorption the at least one complexing agent (b) wherein p′=0 and X is chosen from radicals CW′, and then, in a second stage, applying to the keratin material the at least one compound (a) wherein p≠0 and Y is chosen from groups Z.

33. The process for treating a keratin material according to claim 16, comprising, in a first stage, depositing onto the keratin material by adsorption the at least one complexing agent (b) wherein p′=0 and X is chosen from radicals CW′, and then, in a second stage, applying to the keratin material the at least one compound (a) wherein p≠0 and Y is chosen from radicals BW.

34. The process for treating a keratin material according to claim 16, comprising, in a first stage, depositing onto the keratin material by covalent attachment the at least one compound (a) wherein p≠0 and Y is chosen from groups Z, and then, in a second stage, applying to the keratin material the at least one complexing agent (b) wherein p′=0 and X is chosen from groups Z′.

35. The process for treating a keratin material according to claim 16, comprising, in a first stage, depositing onto the keratin material by covalent attachment the at least one compound (a) wherein p≠0 and Y is chosen from radicals BW, and then, in a second stage, applying to the keratin material the at least one complexing agent (b) wherein p′=0 and X is chosen from groups Z′.

36. The process for treating a keratin material according to claim 16, comprising, in a first stage, depositing onto the keratin material by covalent attachment the at least one compound (a) wherein p≠0 and Y is chosen from groups Z, and then, in a second stage, applying to the keratin material the at least one complexing agent (b) wherein p′=0 and X is chosen from radicals CW′.

37. The process for treating a keratin material according to claim 16, comprising, in a first stage, depositing onto the keratin material by covalent attachment the at least one compound (a) wherein p≠0 and Y is chosen from radicals BW, and then, in a second stage, applying to the keratin material the at least one complexing agent (b) wherein p′=0 and X is chosen from radicals CW′.

38. The process for treating a keratin material according to claim 16, comprising, in a first stage, depositing onto the keratin material by covalent attachment the at least one complexing agent (b) wherein p′≠0 and X is chosen from groups Z′, and then, in a second stage, applying to the keratin material the at least one compound (a) wherein p=0 and Y is chosen from groups Z.

39. The process for treating a keratin material according to claim 16, comprising, in a first stage, depositing onto the keratin material by covalent attachment the at least one complexing agent (b) wherein p′≠0 and X is chosen from groups Z′, and then, in a second stage, applying to the keratin material the at least one compound (a) wherein p=0 and Y is chosen from radicals BW.

40. The process for treating a keratin material according to claim 16, comprising, in a first stage, depositing onto the keratin material by covalent attachment the at least one complexing agent (b) wherein p′≠0 and X is chosen from radicals CW′, and then, in a second stage, applying to the keratin material the at least one compound (a) wherein p=0 and Y is chosen from groups Z.

41. The process for treating a keratin material according to claim 16, comprising, in a first stage, depositing onto the keratin material by covalent attachment the at least one complexing agent (b) wherein p′≠0 and X is chosen from radicals CW′, and then, in a second stage, applying to the keratin material the at least one compound (a) wherein p=0 and Y is chosen from radicals BW.

42. The process for treating a keratin material according to claim 16, comprising, in a first stage, depositing onto the keratin material by covalent attachment the at least one compound (a) wherein p≠0 and Y is chosen from groups Z, and then, in a second stage, applying to the keratin material the at least one complexing agent (b) wherein p′≠0 and X is chosen from groups Z′.

43. The process for treating a keratin material according to claim 16, comprising, in a first stage, depositing onto the keratin material by covalent attachment the at least one compound (a) wherein p≠0 and Y is chosen from radicals BW, and then, in a second stage, applying to the keratin material the at least one complexing agent (b) wherein p′≠0 and X is chosen from groups Z′.

44. The process for treating a keratin material according to claim 16, comprising, in a first stage, depositing onto the keratin material by covalent attachment the at least one compound (a) wherein p≠0 and Y is chosen from groups Z, and then, in a second stage, applying to the keratin material the at least one complexing agent (b) wherein p′≠0 and X is chosen from radicals CW′.

45. The process for treating a keratin material according to claim 16, comprising, in a first stage, depositing onto the keratin material by covalent attachment the at least one compound (a) wherein p≠0 and Y is chosen from radicals BW, and then, in a second stage, applying to the keratin material the at least one complexing agent (b) wherein p′≠0 and X is chosen from radicals CW′.

46. The process for treating a keratin material according to claim 16, comprising, in a first stage, depositing onto the keratin material by covalent attachment the at least one complexing agent (b) wherein p′≠0 and X is chosen from groups Z′, and then, in a second stage, applying to the keratin material the at least one compound (a) wherein p≠0 and Y is chosen from groups Z.

47. The process for treating a keratin material according to claim 16, comprising, in a first stage, in depositing onto the keratin material by covalent attachment the at least one complexing agent b) wherein p′≠0 and X is chosen from groups Z′, and then, in a second stage, applying to the keratin material the at least one compound (a) wherein p≠0 and Y is chosen from radicals BW.

48. The process for treating a keratin material according to claim 16, comprising, in a first stage, depositing onto the keratin material by covalent attachment the at least one complexing agent (b) wherein p′≠0 and X is chosen from radicals CW′, and then, in a second stage, applying to the keratin material the at least one compound (a) wherein p≠0 and Y is chosen from groups Z.

49. The process for treating a keratin material according to claim 16, comprising, in a first stage, depositing onto the keratin material by covalent attachment the at least one complexing agent (b) wherein p′≠0 and X is chosen from radicals CW′, and then, in a second stage, applying to the keratin material the at least one compound (a) wherein p≠0 and Y is chosen from radicals BW.

50. The process for treating a keratin material according to claim 16, comprising simultaneously depositing onto the keratin material by absorption the at least one compound (a) wherein p=0 and Y is chosen from groups Z and the at complexing agent (b) wherein p′=0 and X is chosen from groups Z′.

51. The process for treating a keratin material according to claim 16, comprising simultaneously depositing onto the keratin material by absorption the at least one compound (a) wherein p=0 and Y is chosen from groups BW and the at least one complexing agent (b) wherein p′=0 and X is chosen from groups Z′.

52. The process for treating a keratin material according to claim 16, comprising simultaneously depositing onto the keratin material by absorption the at least one compound (a) wherein p=0 and Y is chosen from groups Z and the at least one complexing agent (b) wherein p′=0 and X is chosen from radicals CW′.

53. The process for treating a keratin material according to claim 16, comprising simultaneously depositing onto the keratin material by absorption the at least one compound (a) wherein p=0 and Y is chosen from radicals BW and the at least one complexing agent (b) wherein p′=0 and X is chosen from radicals CW′.

54. The process for treating a keratin material according to claim 16, comprising simultaneously depositing onto the keratin material the at least one compound (a) wherein p=0 and Y is chosen from groups Z and the at least one complexing agent (b) wherein p′≠0 and X is chosen from groups Z′.

55. The process for treating a keratin material according to claim 16, comprising simultaneously depositing onto the keratin material the at least one compound (a) wherein p=0 and Y is chosen from radicals BW and the at least one complexing agent (b) wherein p′≠0 and X is chosen from groups Z′.

56. The process for treating a keratin material according to claim 16, comprising simultaneously depositing onto the keratin material the at least one compound (a) wherein p=0 and Y is chosen from groups Z and the at least one complexing agent (b) wherein p′≠0 and X is chosen from radicals CW′.

57. The process for treating a keratin material according to claim 16, comprising simultaneously depositing onto the keratin material the at least one compound (a) wherein p=0 and Y is chosen from radicals BW and the at least one complexing agent (b) wherein p′≠0 and X is chosen from radicals CW′.

58. The process for treating a keratin material according to claim 16, comprising simultaneously depositing onto the keratin material the at least one compound (a) wherein p≠0 and Y is chosen from groups Z and the at least one complexing agent (b) wherein p′=0 and X is chosen from groups Z′.

59. The process for treating a keratin material according to claim 16, comprising simultaneously depositing onto the keratin material the at least one compound (a) wherein p≠0 and Y is chosen from radicals BW and the at least one complexing agent (b) wherein p′=0 and X is chosen from groups Z′.

60. The process for treating a keratin material according to claim 16, comprising simultaneously depositing onto the keratin material the at least one compound (a) wherein p≠0 and Y is chosen from groups Z and the at least one complexing agent (b) wherein p′=0 and X is chosen from radicals CW′.

61. The process for treating a keratin material according to claim 16, comprising simultaneously depositing onto the keratin materials the at least one compound (a) wherein p≠0 and Y is chosen from radicals BW and the at least one complexing agent (b) wherein p′=0 and X is chosen from radicals CW′.

62. The process for treating a keratin material according to claim 16, comprising simultaneously depositing onto the keratin material the at least one compound (a) wherein p≠0 and Y is chosen from groups Z and the at least one complexing agent (b) wherein p′≠0 and X is chosen from groups Z′.

63. The process for treating a keratin material according to claim 16, comprising simultaneously depositing onto the keratin material the at least one compound (a) wherein p≠0 and Y is chosen from radicals BW and the at least one complexing agent (b) wherein p′≠0 and X is chosen from groups Z′.

64. The process for treating a keratin material according to claim 16, comprising simultaneously depositing onto the keratin material the at least one compound (a) wherein p≠0 and Y is chosen from groups Z and the at least one complexing agent (b) wherein p′≠0 and X is chosen from radicals CW′.

65. The process for treating a keratin material according to claim 16, comprising simultaneously depositing onto the keratin material the at least one compound (a) in which p≠0 and Y is chosen from radicals BW and the at least one compound (b) wherein p′≠0 and X is chosen from radicals CW′.

66. The process for treating a keratin material according to claim 16, further comprising at least one subsequent step wherein the at least one compound (a) and/or the at least one complexing agent (b) is applied to the keratin material.

67. A multicompartment kit for treating a keratin material, comprising:
a first compartment comprising at least one compound (a) chosen from compounds of formula (I):
L—(A)ₙ—(Y)ₘ—(α)ₚ (I)
m is a number ranging from 0 to 100;
n is equal to 0 or 1;
p is a number ranging from 0 to 100;
α is chosen from anchoring groups allowing covalent attachment to a keratin material;
L is chosen from residues having complexing activity relative to the at least one complexing agent (b), provided that L is not biotin;
A is chosen from linear and branched, saturated and unsaturated divalent radicals comprising from 1 to 100 carbon atoms, optionally interrupted with at least one hetero atom and optionally substituted with at least one group chosen from hydroxyl, amino, halogen, aryl, phosphate, phosphonate, sulphate, sulphonate, carboxyl, alkoxycarbonyl and alkoxy groups;
Y is chosen from groups Z, wherein Z is chosen from carboxyl, mercaptan, alkoxycarbonyl, amino, ammonio, phosphite, phosphate, phosphonate, sulphate sulphite, and sulphonate and radicals BW, wherein W is chosen from residues with cosmetic activity and B is chosen from species for fixing the residue W to L or to A;
B is chosen from amine, imine, amide, ester, disulphide, thioester, urethane, urea, ether, thioether, azo, methine and epoxy groups, aromatic and heterocyclic residues and dialkylpolysiloxane residues; and
W is derived from at least one of UV-screening molecules, hydrating and emollient molecules, conditioners, antistatic agents, antiperspirants, fragrancing materials, reducing molecules, oxidizing molecules, coloring molecules, antimicrobial agents, antidandruff agents, mineral and organic particulates onto which are optionally adsorbed at least one polymer, and anionic and nonionic, amphoteric and cationic film-forming agents onto which are optionally adsorbed organic and/or mineral particles; and
a second comparment comprising at least one complexing agent (b) chosen from compounds of formula (II):
D(A′)_{n′}(X)_{m′}(α′)_{p′} (II)
wherein:
α′ is chosen from anchoring groups allowing covalent attachment to a keratin material;
X is chosen from Z′ and radicals CW′;
Z′ is chosen from carboxyl, mercaptan, alkoxycarbonyl, amino, ammonio, phosphite, phosphate, phosphonate, sulphate sulphite, and sulphonate radicals;
m′ is a number ranging from 0 to 100;
n′ is equal to 0 or 1;
p′ is a number ranging from 0 to 100;
A′ is chosen from linear and branched, saturated and unsaturated divalent radicals comprising from 1 to 100 carbon atoms, optionally interrupted with at least one hetero atom and optionally substituted with at least one group chosen from hydroxyl, amino, halogen, aryl, phosphate, phosphonate, sulphate, sulphonate, carboxyl, alkoxycarbonyl and alkoxy groups;
C is chosen from moieties for attaching the residue W to the active complexing component D; and
W′ is derived from at least one of UV-screening molecules, hydrating and emollient molecules, conditioners, antistatic agents, antiperspirants, fragrancing materials, reducing molecules, oxidizing molecules, coloring molecules, antimicrobial agents, antidandruff agents, mineral and organic particulates onto which are optionally adsorbed at least one polymer, and anionic and nonionic, amphoteric and cationic film-forming agents onto which are optionally adsorbed organic and/or mineral particles; and
D is such that the combination D(A′)_{n′}(X)_{m′}(α′)_{p′} can form with the at least one compound (a) a complex whose dissociation constant is less than 0.1 at a temperature of 25° C.

68. The kit for treating a keratin material according to claim 67, comprising, in the first compartment, the at least one compound (a) wherein p=0 and Y is chosen from groups Z and, in the second compartment, the at least one complexing agent (b) wherein p′=0 and X is chosen from groups Z′.

69. The kit for treating a keratin material according to claim 67, comprising, in the first compartment, the at least one compound (a) wherein p=0 and Y is chosen from groups BW and, in the second compartment, the at least one complexing agent (b) wherein p′=0 and X is chosen from groups Z′.

70. The kit for treating a keratin material according to claim 67, comprising, in the first compartment, the at least one compound (a) wherein p=0 and Y is chosen from groups Z and, in the second compartment, the at least one complexing agent (b) whereom p′=0 and X is chosen grom radicals CW′.

71. The kit for treating a keratin material according to claim 67, comprising, in a first compartment, the at least one compound (a) wherein p=0 and Y is chosen from groups BW and, in the second compartment, the at least one complexing agent (b) wherein p′=0 and X is chosen from radicals CW′.

72. The kit for treating a keratin material according to claim 67, comprising, in the first compartment, the at least one compound (a) wherein p=0 and Y is chosen from groups Z and, in the second compartment, the at least one complexing agent (b) wherein p′≠0 and X is chosen from groups Z′.

73. The kit for treating a keratin material according to claim 67, comprising, in the first compartment, the at least one compound (a) wherein p=0 and Y is chosen from groups BW and, in the second compartment, the at least one complexing agent (b) wherein p′≠0 and X is chosen from groups Z′.

74. The kit for treating a keratin material according to claim 67, comprising, in the first compartment, the at least one compound (a) wherein p=0 and Y is chosen from groups Z and, in the second compartment, the at least one complexing agent (b) wherein p′≠0 and X is chosen from radicals CW′.

75. The kit for treating a keratin material according to claim 67, comprising, in the first compartment, the at least one compound (a) wherein p=0 and Y is chosen from groups BW and, in the second compartment, the at least one complexing agent (b) wherein p′≠0 and X is chosen from radicals CW′.

76. The kit for treating a keratin material according to claim 67, comprising, in the first compartment, the at least one compound (a) wherein p≠0 and Y is chosen from groups Z and, in the second compartment, the at least one complexing agent (b) wherein p′=0 and X is chosen from groups Z′.

77. The kit for treating a keratin material according to claim 67, comprising, in the first compartment, the at least one compound (a) wherein p≠0 and Y is chosen from groups BW and, in the second compartment, the at least one complexing agent (b) wherein p′=0 and X is chosen from groups Z′.

78. The kit for treating a keratin material according to claim 67, comprising, in the first compartment, the at least one compound (a) wherein p≠0 and Y is chosen from groups Z and, in the second compartment, the at least one complexing agent (b) wherein p′=0 and X is chosen from radicals CW′.

79. The kit for treating a keratin material according to claim 67, comprising, in the first compartment, the at least one compound (a) wherein p≠0 and Y is chosen from groups BW and, in the second compartment, the at least one complexing agent (b) wherein p′=0 and X is chosen from radicals CW′.

80. The kit for treating a keratin material according to claim 67, comprising, in the first compartment, the at least one compound (a) wherein p≠0 and Y is chosen from groups Z and, in the second compartment, the at least one complexing agent (b) wherein p′≠0 and X is chosen from groups Z′.

81. The kit for treating a keratin material according to claim 67, comprising, in the first compartment, the at least one compound (a) wherein p≠0 and Y is chosen from radicals BW and, in the second compartment, the at least one complexing agent (b) wherein p′≠0 and X is chosen from groups Z′.

82. The kit for treating a keratin material according to claim 67, comprising, in the first compartment, the at least one compound (a) wherein p≠0 and Y is chosen from groups Z and, in the second compartment, the at least one complexing agent (b) wherein p′≠0 and X is chosen from radicals CW′.

83. The kit for treating a keratin material according to claim 67, comprising, in the first compartment, the at least one compound (a) wherein p≠0 and Y is chosen from radicals BW and, in the second compartment, the at least one complexing agent (b) wherein p′≠0 and X is chosen from radicals CW′.

84. A process for treating a keratin material, comprising:
applying to a keratin material at least one cosmetic treatment agent chosen from reducing agents, oxidizing agents for permanently reshaping the hair, oxidation dyes, bleaching agents, shampoos, styling compositions;
then applying, sequentially or simultaneously, to the keratin material a sufficient amount for treatment of at least one compound (a) and at least one complexing agent (b) as defined below,
wherein said at least one compound (a) is chosen from compounds of formula (I):
L—(A)ₙ—(Y)ₘ—(α)ₚ (I)
m is a number ranging from 0 to 100;
n is equal to 0 or 1;
p is a number ranging from 0 to 100;
α is chosen from anchoring groups allowing covalent attachment to a keratin material;
L is chosen from residues having complexing activity relative to the at least one complexing agent (b), provided that L is not biotin;
A is chosen from linear and branched, saturated and unsaturated divalent radicals comprising from 1 to 100 carbon atoms, optionally interrupted with at least one hetero atom and optionally substituted with at least one group chosen from hydroxyl, amino, halogen, aryl, phosphate, phosphonate, sulphate, sulphonate, carboxyl, alkoxycarbonyl and alkoxy groups;
Y is chosen from groups Z, wherein Z is chosen from carboxyl, mercaptan, alkoxycarbonyl, amino, ammonio, phosphite, phosphate, phosphonate, sulphate sulphite, and sulphonate and radicals BW, wherein W is chosen from residues with cosmetic activity and B is chosen from species for fixing the residue W to L or to A;
B is chosen from amine, imine, amide, ester, disulphide, thioester, urethane, urea, ether, thioether, azo, methine and epoxy groups, aromatic and heterocyclic residues and dialkylpolysiloxane residues; and
W is derived from at least one of UV-screening molecules, hydrating and emollient molecules, conditioners, antistatic agents, antiperspirants, fragrancing materials, reducing molecules, oxidizing molecules, coloring molecules, antimicrobial agents, antidandruff agents, mineral and organic particulates onto which are optionally adsorbed at least one polymer, and anionic and nonionic, amphoteric and cationic film-forming agents onto which are optionally adsorbed organic and/or mineral particles; and
and wherein said at least one complexing agent (b) is chosen from compounds of formula (II):
D(A′)_{n′}(X)_{m′}(α′)_{p′} (II)
wherein:
α′ is chosen from anchoring groups allowing covalent attachment to a keratin material;
X is chosen from groups Z′ and radicals CW′;
Z′ is chosen from carboxyl, mercaptan, alkoxycarbonyl, amino, ammonio, phosphite, phosphate, phosphonate, sulphate sulphite, and sulphonate radicals;
m′ is a number ranging from 0 to 100;
n′ is equal to 0 or 1;
p′ is a number ranging from 0 to 100;
A′ is chosen from linear and branched, saturated and unsaturated divalent radicals comprising from 1 to 100 carbon atoms, optionally interrupted with at least one hetero atom and optionally substituted with at least one group chosen from hydroxyl, amino, halogen, aryl, phosphate, phosphonate, sulphate, sulphonate, carboxyl, alkoxycarbonyl and alkoxy groups;
W′ is derived from at least one of UV-screening molecules, hydrating and emollient molecules, conditioners, antistatic agents, antiperspirants, fragrancing materials, reducing molecules, oxidizing molecules, coloring molecules, antimicrobial agents, antidandruff agents, mineral and organic particulates onto which are optionally adsorbed at least one polymer, and anionic and nonionic, amphoteric and cationic film-forming agents onto which are optionally adsorbed organic and/or mineral particles;
C is chosen from moieties for attaching the residue W to the active complexing component D; and
D is such that the combination D(A′)_{n′}(X)_{m′}(α′)_{p′} can form with the at least one compound (a) a complex whose dissociation constant is less than 0.1 at a temperature of 25° C.
